SEQUENCE LISTING (6 sequences)

SEQ ID NO: 1 - DNA, 9848 bp, Mus musculus
  CDS (329)..(8578)
gctgaagcgtttcctcaagcctgccggggtgggaggagaggaggaggtggtggtggtggaggaggtggaggcagagggtggagagagagaaagcgcacgccgagaggaggtgtgggtgttccgctcccatcctaacggaacgagctccctcttcgcggacatgggattgcccagcggctgctaacccctctcctggtcctgatcccccaaaccggcgtggctccccggtcaccaaggagctgattacaagggaccaggatttgcatccttggctgggcgtccattggctacagagtgcctgacctgggtcaggctttccaacacggacatgtctgacaaaatgtcgagtttcctacatattggagacatttgttctctgtatgcggagggatctacgaatggatttatcagcaccttaggcttggttgatgaccgttgtgttgtacagccagaagccggggaccttaacaatccacccaagaaattcagagactgcctctttaagctatgtcctatgaatcgatactccgcacagaaacagttctggaaagctgctaagcccggggccaacagcactacagatgcagtgctgctcaacaaattgcatcatgctgcagacttggaaaagaagcagaatgagacagaaaacaggaaattgttggggaccgtcatccaatatggcaacgtgatccagctcctgcatttgaaaagcaataaatacctgactgtgaataagaggctcccagccttgctagagaagaatgccatgagggtgacgttggacgaggctggaaatgaagggtcctggttttacattcaaccattttacaagcttcgctccatcggagacagtgtggtcataggcgacaaggtagttttgaatcctgtcaatgctggccagcctctacatgccagcagtcatcagctggtggataacccaggctgcaatgaggtcaactccgtcaactgtaatacaagctggaagatagtgcttttcatgaaatggagtgataacaaagacgacattctcaaaggaggtgatgtggtgaggctcttccatgccgagcaagagaagtttctcacctgtgatgagcaccggaagaagcagcatgtgttcctgaggaccaccggcaggcagtcagccacgtcggccaccagttctaaagccctgtgggaagtggaggtagtccagcacgacccatgtcggggtggagctgggtactggaatagcctcttccggttcaagcacctggctacagggcattacttggctgcagaggtagaccctgactttgaggaagaatgcctggagtttcagccctcagtggaccctgatcaggatgcatctcggagtaggttgagaaacgcgcaagaaaaaatggtatactctctggtctccgtgcctgaaggcaacgacatctcctccatctttgagctagaccccacgactctgcgtggaggtgacagccttgtcccaaggaactcctatgtccgtctcagacacctgtgcaccaacacctgggtacacagcacaaacatccccatcgacaaggaagaggagaagcctgtgatgctgaaaattggtacctctcccctgaaggaggacaaggaagcatttgccatagttcctgtttcccctgctgaggttcgggacctggactttgccaatgatgccagcaaggtgctgggctccatcgctgggaagttggaaaagggcaccatcacccagaatgagagaaggtctgtcacgaagcttttggaagacttggtttactttgtcacgggtggaactaactctggccaagacgtgcttgaagttgtcttctctaagcccaatcgagagcggcagaagctgatgagggaacagaatattctcaagcagatcttcaagctgttgcaggcccccttcacggactgcggggatggcccgatgcttcggctggaggagctgggggatcagcgccatgctcctttcagacatatttgccgactctgctacagggtcctgcgacactcacagcaagactacaggaagaaccaggagtacatagccaagcagtttggcttcatgcagaagcagattggctatgacgtgctggccgaagacaccatcactgccctgctccacaacaaccggaaactcctggagaagcacatcaccgcggcagagattgacacgtttgtcagcctggtgcgaaagaacagggagcccaggttcttggattacctctctgacctctgcgtatccatgaacaagtcaatccctgtgacacaggagctcatctgtaaagctgtgctcaatcccaccaatgctgacatcctgattgagaccaagctggttctttctcgttttgagtttgaaggcgtttccactggagagaatgctctggaagccggggaggatgaggaagaggtgtggctgttctggagggacagcaacaaagagatccgtagtaagagtgtccgggaattggcgcaagatgctaaagagggacagaaggaagacagggacatcctcagctactacagatatcagctgaacctctttgcaaggatgtgtctggaccgccagtacctggccatcaatgaaatctccgggcagctggatgttgatctcattctccgctgcatgtctgacgagaacctcccctacgacctcagggcatccttttgccgcctcatgcttcacatgcatgtggaccgagatccccaagagcaggtgacacctgtgaaatatgcccgactgtggtcagaaattccctctgagatcgccattgatgactatgacagcagtggaacatccaaagatgaaattaaggagaggtttgcacagacgatggagtttgtggaggagtacctaagagatgtggtttgtcaaagattccccttctctgataaggagaaaaataagctcacgtttgaggttgtgaacttagccaggaatctcatatactttggtttctacaacttttctgaccttctccgattaaccaagatcctcttggcaatcttagactgtgtccatgtgaccactatcttccccattagcaagatgacaaaaggagaagagaataaaggcagtaacgtgatgaggtctatccatggcgttggggagctgatgacccaggtggtgctgcggggaggaggcttcttgcccatgactcccatggctgcggcccctgaaggaaatgtgaagcaggcagagccagagaaagaggacatcatggtcatggacaccaagttgaagatcattgaaatactccagtttattttgaatgtgagattggattataggatctcctgcctcctgtgtatatttaagcgagagtttgatgaaagcaattcccagtcatcagaaacatcctccggaaacagcagccaggaagggccaagtaatgtgccaggtgctcttgactttgaacacattgaagaacaagcggaaggcatctttggaggaagtgaggagaacacacctttggacctggatgaccatggtggcagaaccttcctcagggtcctgctccacttgacaatgcatgactacccacccctggtgtctggggccctgcagctcctctttcggcacttcagccagaggcaggaggtccttcaggccttcaaacaggttcaactgctggttactagccaagatgtggacaactacaaacagatcaagcaagacttggaccaactaaggtccattgtggagaagtctgagctctgggtgtacaaaggccaaggtcccgatgagcctatggacggagcctccggtgaaaatgagcataagaaaaccgaggaggggacgagcaagccactgaagcacgagagcaccagcagctacaactaccgagtggtgaaagagattttgattcgacttagcaagctctgcgtgcaggagagcgcgtcggtgaggaagagccggaagcagcagcaacgactgctgaggaacatgggcgcacacgctgtggtgctggagctgctgcagatcccctacgagaaggccgaagacacaaagatgcaagagatcatgcggctggctcatgaatttttgcagaatttctgtgcaggcaaccagcagaatcaagctttgctgcataaacacataaacctgtttctcaagccagggatcctggaggcagtgacgatgcagcacatcttcatgaacaacttccagctgtgcagtgagatcaacgagagagtggtccagcactttgttcactgcatagagacccacggtcgaaacgtccagtatatcaagtttctccagacgattgtcaaggcagaagggaaattcattaaaaagtgccaagacatggtcatggctgagcttgtcaactctggagaggacgtcctcgtgttctacaatgacagagcctctttccagactctgatccagatgatgcggtccgagcgtgaccggatggatgagaacagccctctcatgtaccacatccatctggtggagctcttggccgtgtgcacagagggcaagaatgtgtacacggagatcaagtgcaactccttgctcccgctcgatgacatcgttcgtgtggtcactcatgaagactgcatccccgaggttaagatcgcttacattaacttcctgaatcactgctatgtggatacggaggtggagatgaaggagatttacacaagcaaccacatgtggaagttgtttgagaatttcctcgtggacatctgcagggcctgtaacaacacaagcgacaggaagcacgcagactccattctggagaagtacgtcactgaaatcgtgatgagcatcgtcaccaccttcttcagctctcccttctcagaccagagcaccactctgcagacccgccagcctgtctttgtgcaactcctgcaaggcgtgttccgagtttaccactgcaactggctgatgccgagccaaaaagcctcggtggagagctgcatccgggtgctctctgacgtagccaagagccgggccatagccattcctgttgacctggacagccaagtcaacaacctcttcctgaagtcccacaacattgtgcagaaaacagccctgaactggcggttatcagcccgaaacgccgctcgcagagactctgtactggcagcatccagagactaccgaaatatcattgagaggttacaggacatcgtgtctgccctagaggaccggctcaggcccctggtgcaggctgagctgtctgtgctcgtggatgttctacacagaccagaactgctcttccccgagaacacggatgccaggaggaaatgtgagagtggaggtttcatctgcaagctaataaaacataccaagcaactgctggaggagaatgaagagaaactatgcattaaagtcttacagaccctcagggaaatgatgaccaaagacagaggctatggagagaagcaaatttccattgatgaatcggaaaatgccgagctgccacaggcaccggaagctgagaactccacagagcaggagcttgaaccaagtccacccctgaggcaactggaagaccataaaaggggtgaggcactccgacaaattttggtcaaccgttactatggaaacatcagaccttcaggaagaagagagagccttaccagctttggcaatggcccactatcaccaggaggacccagcaagcctggtggaggagggggaggtcctggatctagttccacaagcaggggtgagatgagcctggctgaggttcagtgtcacctcgacaaggagggggcctccaacctggtcatcgatctcataatgaatgcatccagtgaccgagtattccatgaaagcattctgctggccatcgcacttctggaaggaggcaacaccaccatccagcactcgtttttctgccggctgacagaagataagaaatcagagaagttcttcaaggttttttacgatcgaatgaaggtggcccagcaggaaatcaaggcgacagtgacagtgaacaccagcgacttgggaaacaaaaagaaagatgatgaagtggacagggatgccccgtctcggaagaaagccaaagagcccacaacacagataacagaagaggtccgggatcagctcctggaagcatctgctgccaccaggaaagcctttaccaccttccggagggaggccgaccctgatgaccattaccagtctggggagggcacccaggctacaaccgacaaagccaaggatgacctagagatgagcgctgtcatcaccatcatgcagcctatcctgcgcttcctgcagctgctgtgtgaaaaccacaaccgagatctgcagaatttccttcgttgccaaaataataagaccaactacaatttggtgtgtgagacactgcagtttctggactgtatttgtgggagcacaaccggaggccttggtcttcttggactgtacataaatgaaaagaatgtagcacttatcaaccaaaccctggagagtctgacggagtactgtcaagggccttgccatgagaaccagaactgcatcgccacccacgagtccaatggcatcgatatcatcacagccctcatcctcaatgatatcaaccctctgggaaagaagcggatggacctggtgttagaactgaagaacaatgcttcgaagctgctactggccatcatggaaagcagacacgatagtgaaaatgcagagaggatcctgtacaacatgaggcccaaggagctggtggaagtgatcaagaaggcctacatgcaaggtgaagtggaatttgaggatggggagaacggtgaggatggagctgcctcacccaggaacgtgggccacaacatctacatcctcgctcaccagttggctcggcataacaaagaacttcaaaccatgctgaaacctggaggccaggtggatggggatgaagctctggagttctacgcgaagcacacagcacaaattgagattgtcagactggaccggacaatggaacagatcgtcttccctgtgcccagcatctgtgaattcctgactaaggaatcgaaacttcgaatatattacaccacagagcgggatgagcaaggtagcaagatcaatgacttcttcctgcgctccgaggacctctttaacgagatgaactggcagaagaaacttcgagcccagcctgtcttgtactggtgtgcccgaaacatgtctttctggagcagcatctccttcaacctggccgtcctgatgaacctgctggtggcgtttttctatccatttaaaggagtgaggggaggaacactagagccacactggtcaggcctcctgtggacagccatgctcatctctctggccattgtcattgctctgcccaagccccacggcatccgggccttaattgcttctacaatcctacgactgatattttcagttgggttgcagcccacactgtttctgctgggagctttcaatgtctgcaataaaatcatcttcctgatgagctttgtgggcaactgtgggaccttcaccagaggctaccgggccatggttctggatgtggagttcctctatcatttgctgtatctactcatctgtgccatgggcctcttcgtacatgagttcttctatagcttgctgctttttgatttagtgtacagagaggagactttgcttaatgtcattaaaagtgtcacccgcaatggacggtccatcatcttgacagcggtcctggctctgatcctggtttacctgttctcaattgtgggctatctgttcttcaaggatgactttatcttggaagtagataggttgcccaatgaaacagctgttccagaaactggcgagagtttggccaacgatttcctgtactctgatgtgtgcagggtagagacgggggagaactgcacctctcctgcacccaaagaagagctgctccctgccgaagaaacggaacaggataaggaacacacgtgtgagaccctgctcatgtgcatcgtcactgttctgagtcacgggctgcggagtgggggaggggtaggagacgtgctcaggaagccatccaaagaggagcctctgtttgctgcaagggtgatctacgacctcctcttcttcttcatggtcatcatcatcgtcctgaacctgattttcggggtcatcatcgacacctttgctgacctgaggagtgagaagcaaaagaaggaggagatcttaaaaaccacgtgcttcatctgcggcttggaaagggacaagtttgacaataagactgtcacctttgaagagcacatcaaggaagaacacaacatgtggcactatctgtgcttcatcgtgctggtgaaagtgaaggactccacagagtacaccgggcctgagagttacgtggcagagatgatcagggaaagaaaccttgattggttcctcagaatgagagccatgtccctggtcagcagcgattctgaaggggaacagaacgagctgaggaacctgcaggagaagctggagtctaccatgaagctggtcaccaatctttctggccagctgtcagaactaaaggaccagatgacagaacagaggaagcagaaacaaagaatcggccttctaggacatcctcctcacatgaatgtcaacccacagcagccggcctaggcaaatgaggcagagggactctgctcagccctctgtatatcactgtcagggtgggtacggctcattggttctgatttgcccactaagggtacatgtgcgcttagtacatttgtaaatactcagttttgtattgtatgtatatgattgctattctcagaggtttggactttcgtattgtaattagctctgttggcatggtgacttgtcactcctgccaaaaatattaaaaatgccttttttggaaggactacagaaagtacctgatttgcacttgaaccagattatagatttaaaagtatatgacatgtattttgtatttaaaactagaatagccagtatttatgttttttataaaactgtgcaatacaaattatgcaatcaccataactttgtaactcctgagtgtcctaagggagtacacatctttgaagctgatttgttgatactcgtgtaataaatggttaaatatcaaatgctgctgctgctgccaaaattatattaatagcgagtttctggcccctgggcaattttgtaccttgtaattatcctatggtgatgctgtttctcgttgctaatggcattagtgcccctgtatcctagtgataactccaggtctgtgaaccattcaaacagcattcattttgagaaaagcaactttagtttcaaggataattttaagcttcaaaattaatcatttaaagtgtttctttaagagagccatgttagaggctcacactttagcttgaaaggagttgatgaattaattttttaaagggaactttttacatgacgtttggaataacagcatattgctgaccagtcagtgtcatctcccgggtgaattttgatgtcacgttatagtcaaatgagttagctgatggtttctagattttcttcctctgaaccatgatgcagtaggtaagaagttattatgcgtatatacatatatacattcatatacgacaaagtaggagctgtccccttaggatgcatagctgcccctagggtacgtagctgaacactgacaatggcgttcttctgaaagagccacgtttgggttttatttctttgtcacatgatttcttttctggatgggtgcaaagtatcacaggaagtgttttctctctgtcgccttgttttgtacctgggtctcgctttactagaccgtctctgcacaaaagtttaaaaactgaaccgtatgcagagttccgaagcaagtcaagtttgtaaatgcatacctaaaaatatttaataaacgatgcagaatcct

SEQ ID NO: 2 - protein, 2749 aa, Mus musculus
MSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSTGENALEAGEDEEEVWLFWRDSNKEIRSKSVRELAQDAKEGQKEDRDILSYYRYQLNLFARMCLDRQYLAINEISGQLDVDLILRCMSDENLPYDLRASFCRLMLHMHVDRDPQEQVTPVKYARLWSEIPSEIAIDDYDSSGTSKDEIKERFAQTMEFVEEYLRDVVCQRFPFSDKEKNKLTFEVVNLARNLIYFGFYNFSDLLRLTKILLAILDCVHVTTIFPISKMTKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQSSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDEPMDGASGENEHKKTEEGTSKPLKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLKPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSILEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHNIVQKTALNWRLSARNAARRDSVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKQISIDESENAELPQAPEAENSTEQELEPSPPLRQLEDHKRGEALRQILVNRYYGNIRPSGRRESLTSFGNGPLSPGGPSKPGGGGGGPGSSSTSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQSGEGTQATTDKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQTMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLLICAMGLFVHEFFYSLLLFDLVYREETLLNVIKSVTRNGRSIILTAVLALILVYLFSIVGYLFFKDDFILEVDRLPNETAVPETGESLANDFLYSDVCRVETGENCTSPAPKEELLPAEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIRERNLDWFLRMRAMSLVSSDSEGEQNELRNLQEKLESTMKLVTNLSGQLSELKDQMTEQRKQKQRIGLLGHPPHMNVNPQQPA

SEQ ID NO: 3 - DNA, 8347 bp, Mus musculus
  CDS (35)..(8140)
gacagcagggacagccacaggagcgtgaagcagcatgtctgacaaaatgtccagcttcctctacattggggacatcgtgtccctgtacgcggagggctcggtcaatggcttcatcagcaccctggggttggtggatgaccgttgtgtggtgcacccagaggcgggagaccttgccaacccacccaagaagttcagagactgcctgttcaaggtgtgccctatgaatcgatactctgcccagaagcaatactggaaagccaagcaggcgaaacaggggaaccatacagaggctgccctgctgaagaagctccagcatgctgcagagctggaacaaaaacagaatgagtcggagaacaggaaacttttgggagaaatcgtgaaatacagcaacgttatccaactgctgcacataaaaagcaacaagtacctgacggtgaacaagaggttacctgccttgctggagaagaatgccatgcgagtgtcactggatgctgcagggaacgaaggctcctggttctacatccatccgttctggaagctgagaagcgagggtgataatatcgtcgtaggagataaagtcgttctgatgcctgtaaatgctgggcagcccctgcatgccagcaacgtggagctcttggacaacccaggctgcaaagaggtgaatgctgttaattgtaacactagctggaaaatcactttattcatgaaattcagttcctatcgagaggatgtattaaaggggggtgatgttgtgagactctttcatgcggaacaagagaaatttctgacctgtgatgactatgagaaaaaacagcacattttccttcggacgaccttgcgtcaatcagcaacttcggccactagttctaaagcactctgggaaatagaggtggttcaccacgatccatgccgcggaggtgcgggacagtggaacagcctgttcaggtttaagcatcttgccactgggaactacttagctgcagagcttaatcctgactatcgagatgctcaaaatgaaggaaaaaatgtgaaagacggggagattccaacaccaaagaaaaaacgccaggcaggggagaagatcatgtacaccctggtttcagtcccacacggaaatgacatcgcgtccctttttgaacttgatgccacaactctgcagagagccgactgcctggttccgaggaactcctacgtgcggttaaggcacttgtgcaccaacacctgggtaacaagcaccaccatccccatagacacagaggaagagaggcctgtcatgttgaaaattgggacctgccagaccaaggaagacaaagaagcctttgccattgtgtgcgtcccgctgtctgaggtccgtgacttggactttgccaacgatgccaacaaagtgctggccaccacggtgaagaagctggagaatggcagcatcacacagaacgagaggaggtttgtgactaagttgttggaagacctcattttctttgtggctgatgtgaccaacaatgggcaggacgttctggatgtggtcatcaccaagccaaaccgggaacggcagaaactaatgagggaacagaatatactggcacaggtgtttgggatccttaaagcacctttcaaggagaaggctggagaaggttcgatgctgaggctggaggacctgggcgaccagcgctacgccccgtacaagtatgtgctgcgtctctgctaccgtgtgctgcggcattcgcagcaggactacaggaagaaccaggagtacattgctaagaacttctgcgttatgcagtcccagatcggctatgatatcttggcagaagatacgattacagccttgttacacaacaaccgcaaactactggagaaacacatcacagccaaagaaattgagactttcgtcagcctgctcaggaggaaccgggagccgaggtttctggactatttgtctgacctctgtgtgtccaatagcacggccatccccgtcactcaggaactcatctgcaaattcatgctgagccctggcaatgcggacatcctcattcagacaaagctggtgtccatgcaagtggagaaccccatggagagctccatccttcctgatgacattgacgatgaggaggtctggctttactggattgacagcaacaaggagcctcacggcaaggccatcaggcaccttgcccaggaagccagggaaggcaccaaagctgacctggaagttctgacctattacaggtaccagctaaacctctttgcaaggatgtgcttggaccgccagtatctggccatcaaccagatttcaacacagttgtccgtagacctgatcctgcggtgtgtgtctgacgagagtctgccctttgacctcagagcatctttctgccgactcatgctccacatgcacgttgaccgcgacccccaggagtctgtagtgcctgttcgctatgcccgcctctggaccgaaatccccacaaagatcacgattcatgagtatgactccattacagactcttccagaaatgacatgaagaggaagtttgccctgacgatggaatttgtcgaagaatatttgaaagaagttgtaaatcaaccatttccttttggagacaaagagaaaaataaactgacatttgaggtggttcacctagcccggaacctcatctatttcggattctacagcttcagcgagttactgcggctgacgagaactctgctggcgatcctagacatcgtccaggcccccatgtcctcatatttcgaaagattaagcaagttccaagatggaagcaacaatgtcatgaggaccatccacggagtgggagagatgatgacacagatggtgctcagcagaggctccatcttcccagtgagtgtgcccgacgcacagcccatcgtccaccccagcaagcaggcgagccccggcgagcaggaggatgtgacagtgatggacaccaagctgaaggttatcgagattttacagttcatcctgagcgtcaggctggactaccggatctcttacatgctgtccatatacaagaaggaatttggagacaacaacgacaatggtgacccctctgccagcggcaccccagacacactgctaccatcagctcttgtccctgacatcgacgagattgcggctcaggcagaaacaatgttcgctggaagaaaagaaaaaactccagttcagcttgatgatgaaggaggcaggacgttcttacgcgtcctcatccacctgatcatgcacgactatgctccgctgctgtccggagccctgcagctgctcttcaagcatttcagccagagagcagaagtgcttcaggcctttaaacaggtgcagttactagtgtcgaaccaagatgttgataactacaagcaaatcaaggcagacctcgaccaacttcggctgacggtggagaaatctgagctgtgggtagaaaagagcggcagctatgagaatggagatgtgggtgagggccaggccaaaggaggcgaagaggcaaatgaggaatccaacctcttgagcccggtgcaggatggcgcgaagacacctcagatcgacagcaacaagggcaacaactaccggatcgtcaaggagatcttgattaggctgagtaagctctgtgtgcagaacaagaaatgccgtaatcaacaccagcggctgctgaaaaacatgggggctcactccgtggtgctggaccttctgcagatcccctatgagaagaccgacgagaagatgaacgaggtgatggacttagcccacaccttcctgcagaacttctgccgtgggaatccgcagaaccaagttctccttcacaagcatctcaatctgttcctaactcctggcctcctggaggcagaaaccatgcggcacatcttcatgaacaattatcacctgtgcaacgagatcagcgagagggtggtccagcacttcgtgcactgcattgagacacatggccgccatgtggagtacctgaggttcctgcagacgatcgtgaaagcagatggcaaatacgtgaagaaatgccaggacatggtgatgacggagttgatcaatgggggcgaggatgtgttgatattctacaatgacagagcttcatttcccatcctcctcaacatgatgtgctctgagagagcccgtggggacgagagcggccccctcgcctaccacatcacccttgtggagttgctggcagcgtgcaccgaaggcaaaaatgtctacacagaaatcaagtgcaattctctcttgcctctggacgacatagtgagagtggtgacccacgatgactgcattcctgaggtcaaaatcgcgtacgtgaactttgtcaaccattgctacgttgacactgaagtggagatgaaggagatctacacaagtaaccacatttggaagttatttgagaatttcttggtggatatggcgagggtctgtaacaccaccacagacaggaaacacgcagacacctttctggagaggtgtgtgacagagtcagtcatgaacattgtgagcggcttcttcaactcgccattttccgacaacagtaccagcctccagacccaccagccagtctttatccagctgctgcagtctgccttcagaatttacaactgtacctggcccaacccagcccagaaagcctccgtagaatcctgtatcagagccctggctgaagtggccaaaaaccgtgggattgcgatcccagtcgatttggacagccaggtcaacacgctcttcatgaagaaccactcaagtacggtgcagagggcagccatgggctggagactctcagctcgctctggacctcgcttcaaggaagctcttggaggaccagcctgggattacagaaatattattgaaaagttacaggatgtcgtggcctccctggaacagcagttcagccccatgatgcaggccgagttctctgtgcttgtggatgtgctgtacagcccggagctactgttcccagagggaagcgacgccaggatccgatgtggtgccttcatgtccaagctgattaatcacacaaagaaactgatggagaaagaagaaaagctgtgcattaaaatcctccagactttacgggagatgctggagaagaaagacagcttcatggaagagggtagcacattacggcgaatcctcctgaatcggtacttcaagggtgatcatagcattagtgtgaacggacctctgtcaggagcctatgccaagacagcacaagtgggaggaagcttctctggacaggattcagataagaagggcatttcaatgtccgatatccagtgtttgctggacaaagaaggagcatccgaacttgtcatcgatgtcattgtgaacaccaaaaacgacaggatcttttccgaaggaattttgcttggcattgccttgctggaaggaggaaacacacagacccagtattctttctaccagcagttgcatgaacaaaaaaagtcagagaaatttttcaaagtcctttatgatcggatgaaggctgctcagaaagagatcagatccacggtgaccgtcaacaccatagacttagggagcaagaagagagaagaggacagtgacgtcatggccctgggccctcggatgagagtgagagactcatcactacatttgagagaaggaatgaaagggcagttaacagaagcgtcttcagccacatccaaagcatactgtgtgtacagaagagagatggacccggaaatagacacaatgtgcccgggacaggaagcaggaagtgcggaggaaaagtctgcggaggaagttaccatgagcccggccatcaccatcatgcgtcccatcctcaggttcctgcagttactgtgtgagaatcataaccgggagcttcagaacttcttgaggaaccagaacaacaaaacaaactacaaccttgtctgtgaaacgcttcagttcctggactgtatctgcggaagtaccacagggggtctaggcctgctggggctctacatcaatgagaggaatgtagctctggtcaaccagactctggagagcttgaccgagtactgccaaggcccgtgccacgagaaccagacctgtatcgccactcacgaatccaatgggattgatatcatcatcgccttgattctgaatgacatcaaccccctgggcaagtacagaatggacttggtgttgcagctgaagaacaacgcctccaagcttctgctggccattatggagagcagacatgacagtgagaatgcagagagaattcttttcaacatgagacccagggaactggtggatgtcatgaagaatgcctataaccaaggcctggaatgtgaccatggggacgaggagggaggggatgatggtgtttctccaaaagacgttggacacaacatctacattctggcccatcagttggcccgccacaacaaactgctgcagcagatgctcaagccgggatctgacccagaggaaggggatgaagctttgaagtattacgccaaccacaccgcgcagatcgagattgtgcggcatgaccggactatggagcagattgttttcccggtccccaacatctgtgaattcctcactcgggaatccaagtaccgagtgttcaacacaacggagagggatgagcaaggaagcaaggtgaacgacttcttccagcaaacggaagatctctacaacgagatgaaatggcaaaagaagatcagaaacaaccccgccctgttctggttctccaggcacatctcactctgggggagcatctccttcaacctggctgtgttcatcaacctggctgtggctctcttctacccattcggggatgacggtgatgaaggcacactctccccgatgttctcagtcctcctttgggtagcagtggcgatctgcacatctatgctgttcttcttctccaagcccgtgggcatccggccattcctcgtgtctgtcatgctcagatcgatatacaccatcggtctggggccaacgctaatactacttggtgctgccaatctatgtaataaaatcgtgttcctggtgagttttgtgggaaaccgaggcacattcaccagaggctaccgagcagtcattctggacatggcctttctttaccacgtggcctatgtcctggtttgcatgcttggcctctttgtccacgagttcttctacagcttcctgctttttgatttggtatatagagaagagaccctgctgaatgtcatcaaaagcgtcacacggaacggccgctccatcatcctgaccgcggtcttggctcttatcttggtctacctgttctccatcatcggcttccttttcttaaaggacgacttcaccatggaggtggacagactgaaaaacagaactccagtcacaggtaatcacggggttcccactatgaccttatcttccatgatggaaacctgccagaaggaaaactgctcacccacgatcccctcttccaacacagccggcgaggaaggtgaggatggcatagaaaggacatgtgataccctgctcatgtgcattgtcaccgtgctgaaccagggcctcaggaatggcggcggagttggggatgtgctgagacgaccctcgaaagatgagcccttgtttgctgcccgggtagtctacgacctcctgttcttcttcatcgtcatcatcatcgttctcaacctgatctttggagtgatcattgacacgtttgctgacctcagaagtgagaagcagaagaaagaagaaatcctcaagacaacctgcttcatctgtggcctggagagagacaagtttgataacaagacagtgtcctttgaggagcacatcaagtcagagcacaacatgtggcattacctgtacttcatcgtcctggtgaaggtgaaggacccgacagaatacacagggcctgagagctacgtggctcagatgatcacggagaagaatttggactggtttcctcggatgcgagccatgtcacttgtcagcaacgaaggtgacagtgagcaaaatgagatccggaacctgcaggagaagctggagtctacgatgagtctggtgaagcagctgtcagggcagctggcagagctgaaggagcagatgacagaacaaaggaaaaacaagcaaaggctgggctttcttggatcaaacacaccccatgtgaaccatcacatgccaccgcactgacaccatgggggcacgccgtgaccagcctttcatcggatgcctgcctcatcacagagggatggagatggaggggagcgaacagtggctattgttgaaaagctaaaaacaaccaagtgccaagatgctgagccatttagctcccagaacaatccgtaactgtgtttgcacgcttgagaagattcaagcttagaaaaaaaaaaaaaaaaa

SEQ ID NO: 4 - protein, 2701 aa, Mus musculus
MSDKMSSFLYIGDIVSLYAEGSVNGFISTLGLVDDRCVVHPEAGDLANPPKKFRDCLFKVCPMNRYSAQKQYWKAKQAKQGNHTEAALLKKLQHAAELEQKQNESENRKLLGEIVKYSNVIQLLHIKSNKYLTVNKRLPALLEKNAMRVSLDAAGNEGSWFYIHPFWKLRSEGDNIVVGDKVVLMPVNAGQPLHASNVELLDNPGCKEVNAVNCNTSWKITLFMKFSSYREDVLKGGDVVRLFHAEQEKFLTCDDYEKKQHIFLRTTLRQSATSATSSKALWEIEVVHHDPCRGGAGQWNSLFRFKHLATGNYLAAELNPDYRDAQNEGKNVKDGEIPTPKKKRQAGEKIMYTLVSVPHGNDIASLFELDATTLQRADCLVPRNSYVRLRHLCTNTWVTSTTIPIDTEEERPVMLKIGTCQTKEDKEAFAIVCVPLSEVRDLDFANDANKVLATTVKKLENGSITQNERRFVTKLLEDLIFFVADVTNNGQDVLDVVITKPNRERQKLMREQNILAQVFGILKAPFKEKAGEGSMLRLEDLGDQRYAPYKYVLRLCYRVLRHSQQDYRKNQEYIAKNFCVMQSQIGYDILAEDTITALLHNNRKLLEKHITAKEIETFVSLLRRNREPRFLDYLSDLCVSNSTAIPVTQELICKFMLSPGNADILIQTKLVSMQVENPMESSILPDDIDDEEVWLYWIDSNKEPHGKAIRHLAQEAREGTKADLEVLTYYRYQLNLFARMCLDRQYLAINQISTQLSVDLILRCVSDESLPFDLRASFCRLMLHMHVDRDPQESVVPVRYARLWTEIPTKITIHEYDSITDSSRNDMKRKFALTMEFVEEYLKEVVNQPFPFGDKEKNKLTFEVVHLARNLIYFGFYSFSELLRLTRTLLAILDIVQAPMSSYFERLSKFQDGSNNVMRTIHGVGEMMTQMVLSRGSIFPVSVPDAQPIVHPSKQASPGEQEDVTVMDTKLKVIEILQFILSVRLDYRISYMLSIYKKEFGDNNDNGDPSASGTPDTLLPSALVPDIDEIAAQAETMFAGRKEKTPVQLDDEGGRTFLRVLIHLIMHDYAPLLSGALQLLFKHFSQRAEVLQAFKQVQLLVSNQDVDNYKQIKADLDQLRLTVEKSELWVEKSGSYENGDVGEGQAKGGEEANEESNLLSPVQDGAKTPQIDSNKGNNYRIVKEILIRLSKLCVQNKKCRNQHQRLLKNMGAHSVVLDLLQIPYEKTDEKMNEVMDLAHTFLQNFCRGNPQNQVLLHKHLNLFLTPGLLEAETMRHIFMNNYHLCNEISERVVQHFVHCIETHGRHVEYLRFLQTIVKADGKYVKKCQDMVMTELINGGEDVLIFYNDRASFPILLNMMCSERARGDESGPLAYHITLVELLAACTEGKNVYTEIKCNSLLPLDDIVRVVTHDDCIPEVKIAYVNFVNHCYVDTEVEMKEIYTSNHIWKLFENFLVDMARVCNTTTDRKHADTFLERCVTESVMNIVSGFFNSPFSDNSTSLQTHQPVFIQLLQSAFRIYNCTWPNPAQKASVESCIRALAEVAKNRGIAIPVDLDSQVNTLFMKNHSSTVQRAAMGWRLSARSGPRFKEALGGPAWDYRNIIEKLQDVVASLEQQFSPMMQAEFSVLVDVLYSPELLFPEGSDARIRCGAFMSKLINHTKKLMEKEEKLCIKILQTLREMLEKKDSFMEEGSTLRRILLNRYFKGDHSISVNGPLSGAYAKTAQVGGSFSGQDSDKKGISMSDIQCLLDKEGASELVIDVIVNTKNDRIFSEGILLGIALLEGGNTQTQYSFYQQLHEQKKSEKFFKVLYDRMKAAQKEIRSTVTVNTIDLGSKKREEDSDVMALGPRMRVRDSSLHLREGMKGQLTEASSATSKAYCVYRREMDPEIDTMCPGQEAGSAEEKSAEEVTMSPAITIMRPILRFLQLLCENHNRELQNFLRNQNNKTNYNLVCETLQFLDCICGSTTGGLGLLGLYINERNVALVNQTLESLTEYCQGPCHENQTCIATHESNGIDIIIALILNDINPLGKYRMDLVLQLKNNASKLLLAIMESRHDSENAERILFNMRPRELVDVMKNAYNQGLECDHGDEEGGDDGVSPKDVGHNIYILAHQLARHNKLLQQMLKPGSDPEEGDEALKYYANHTAQIEIVRHDRTMEQIVFPVPNICEFLTRESKYRVFNTTERDEQGSKVNDFFQQTEDLYNEMKWQKKIRNNPALFWFSRHISLWGSISFNLAVFINLAVALFYPFGDDGDEGTLSPMFSVLLWVAVAICTSMLFFFSKPVGIRPFLVSVMLRSIYTIGLGPTLILLGAANLCNKIVFLVSFVGNRGTFTRGYRAVILDMAFLYHVAYVLVCMLGLFVHEFFYSFLLFDLVYREETLLNVIKSVTRNGRSIILTAVLALILVYLFSIIGFLFLKDDFTMEVDRLKNRTPVTGNHGVPTMTLSSMMETCQKENCSPTIPSSNTAGEEGEDGIERTCDTLLMCIVTVLNQGLRNGGGVGDVLRRPSKDEPLFAARVVYDLLFFFIVIIIVLNLIFGVIIDTFADLRSEKQKKEEILKTTCFICGLERDKFDNKTVSFEEHIKSEHNMWHYLYFIVLVKVKDPTEYTGPESYVAQMITEKNLDWFPRMRAMSLVSNEGDSEQNEIRNLQEKLESTMSLVKQLSGQLAELKEQMTEQRKNKQRLGFLGSNTPHVNHHMPPH

SEQ ID NO: 5 - DNA, 8773 bp, Mus musculus
  CDS (51)..(8063)
agctcgcttcagggcgacctcgtcgcctcgagccccggagggccgaagccatgaatgaaatgtccagctttcttcacatcggggacattgtgtccctgtacgccgagggctctgtcaatggcttcatcagcactttggggctggtggatgaccgttgtgtggtggaacctgcagctggggacctggacaacccccccaagaagttccgagattgcctcttcaaagtgtgccccatgaaccgctactctgcacagaaacagtactggaaagccaagcagactaaacaggacaaagagaagattgctgatgtggtattgctgcagaagctgcagcacgcagcccagatggagcagaagcaaaatgacacagagaacaagaaggtgcacggggacgtggtgaagtacggcagcgtgattcagcttctgcatatgaagagcaacaaatacttaactgtgaacaagcggctgccagccctgctggagaagaatgccatgcgggtgacgctggatgctacgggcaatgagggctcctggctcttcatccagcccttctggaagcttcggagcaatggggataatgtggtcgtgggggacaaggtgatcctgaatcctgtcaatgccgggcagcccttacatgccagcaactatgagcttagtgataatgctggctgcaaagaggtcaactctgtcaactgcaacaccagctggaagatcaacttattcatgcagttccgggaccacctggaggaggtgctgaaggggggagacgtggtgcgactgttccacgcggagcaggagaagttcctgacctgtgatgagtacaggggcaagctgcaggtgttcctgcggaccacgctccgccagtccgccacctcggccaccagctccaacgcactctgggaggtggaggtggtccaccacgacccctgccgcgggggagccggacactggaacggtctgtacaggttcaagcacctggccacgggcaactatctggccgctgaggaaaaccccagctacaagggtgatgtctcagatcctaaggcagctggactgggggcccagggccgcactggccgcaggaacgcaggcgagaagatcaagtaccgtctggtggctgtgccacatgggaatgacatcgcgtctctcttcgagctggaccccaccacgctgcagaaaaccgactccttcgtgccccggaactcctacgtgcggctgcggcacctctgcaccaacacctggatccagagtaccaacgcgcccatcgacgtggaggaggaacggcctatccggctcatgttgggcacatgccccaccaaggaggacaaagaggcctttgccattgtgtcggttcctgtgtcggagatccgcgacctggactttgccaacgacgccagctccatgctggccagcgccgtggagaaactcaacgaaggcttcatcagccagaacgaccgcaggtttgtcatccagctgttggaggacttggtgttttttgtcagcgatgttcccaacaatgggcagaatgtcctggacatcatggtcaccaagcccaaccgggagcggcagaagctgatgcgggagcagaacatacttaagcagatctttggcatcctgaaggctccattccgtgacaaggggggtgaaggaccactagtgcggctggaggaactgtctgaccagaagaacgccccctaccagtacatgttccgtctctgctaccgggtgttgcggcattctcaggaggactaccgcaagaatcaggagcacatagccaagcagtttgggatgatgcagtcacagattggctatgacatcctggctgaagacaccatcactgccctgctgcacaacaaccggaagcttctagagaaacacatcaccaagacggaggtggagacctttgtcagtctcgtgcgcaagaacagggagcccagattcctggactacctctccgacctgtgcgtgtccaaccgtatcgccatccctgtcacccaagagctgatttgcaaatgtgtgctggaccctaagaacagtgacattcttatccagactgagctgcggccagtgaaggagatggctcagtctcacgagtacctcagcatcgagtactcagaggaggaggtgtggctcacatggacggacaggaacaatgagcaccacgagaagagcgtgaggcagctggcccaggaggcgagggctggcaacgcacacgatgagaacgtgctcagctactacaggtaccagttgaagctgtttgcccgcatgtgcctggaccgccagtacttggccattgatgagatctccaagcagctgggtgtcgagctgcttttcctgtgcatggctgacgagatgctgccctttgacctccgcgcctcattctgccatctgatgctacatgtgcacgtggaccgcgaccctcaggagctggtgacccctgtcaagtttgcccgcctctggacagagatccccacggccatcaccattaaagactatgattccaatctcaacgcgtcgagagatgacaaaaaaaacaagtttgccagcaccatggagttcgtagaagattacctcaacaacgtggtcagcgaggccgtgccctttgccaacgacgagaagaacatactcacctttgaggtggtgagcctggcgcacaatctcatctacttcgggttctacagcttcagcgagctgctgaggctcacacgcacgctactgggcatcatagactgcatccaggcaccggcagccatgctgcaggcctatgaggagcctggtggcaagaatgtgcggaggtccatccagggggtggggcacatgatgtccaccatggtgctgagccgcaagcagtctgtatttggtgcctccagcctgcctgctggagtgggtgtccctgagcagctagacagaagcaaatttgaggacaatgagcacactgtggtgatggagaccaaactgaagatcctggagattctgcagttcatcctgaacgtacgcttagactaccgcatctcctacctgttgtcggtcttcaagaaggagtttgtggaggtgtttcccatgcaggacagcggggcggacggcacggcacctgcctttgattcctccactgccaccatgaatctggaccgcatcggagagcaggcagaggccatgtttggagtagggaagaccagcagcatgctggaggtggacgatgaaggtggccgcatgttcctgcgagtgctgctgcacctgaccatgcacgactacccgtctcttgtctccggtgcgctgcagctgctcttcaaacacttcagccagcgccaggaagccatgcacactttcaagcaggtccagttgctgatctcagcccaggatgtggagaactacaaggtgatcaagtcagagctggaccgactgcggacgatggtggagaagtcagagctgtgggtggacaagaaaggcagcgtcaagggcgaggaggttgaggcaggcgccaccaaggataagaaggagagaccctcggacgaggagggatttctgcagccacacggggagaagagcagcgagaactaccagattgtgaaaggcatcctggagaggctgaacaagatgtgtggggtgggggagcagatgaggaagaagcaacaaaggttgctgaagaacatggacgcccacaaggtcatgctggacctactgcagatcccttacgacaagagcgacaacaagatgctggagatactgcgctacacacaccagttcctacagaagttctgtgctgggaaccctggcaaccaggcccttctgcacaaacacttgcagctcttcctcacgcctgggctcctggaggctgagaccatgcagcacattttcctcaacaactaccagctgtgctctgagatcagcgagccggtgttgcaacactttgtgcacctgctggccacacacgggcgccatgtgcagtacttggacttcctgcacactgtcatcaaggctgagggcaagtacgtgaagaagtgccaggacatgattatgaccgagctgaccaatgcaggtgacgacgtggtggtgttctacaacgacaaggcctctctggctcatctgttggacatgatgaaggcagccagagatggcgtggaggaccacagccctctcatgtatcacatctccctggtggacctgctggctgcctgtgcagagggcaagaacgtctacacagagatcaagtgcacctccctcttgcctctagaggacgtggtgactgtggtcacccacgaggattgtatcacggaggttaaaatggcttatgtgaacttcgtgaatcactgctacgtagacacggaggtggagatgaaggagatctacaccagcaaccacatctggacgctcttcgagaacttcaccctggacatggctctggtctgtaacaagcgggagaagcgcctgtcagaccccacccttgagaagtacgtgctcaccgtggtcctggacaccatcagtgccttcttcagctccccgttttcagagaacagcacatccctacagacgcaccagacaattgtggtccagctgctgcagtccaccacgcggctgctcgagtgtccttggctgcagcagcagcataagggctccgtggaggcctgtgtccgcaccctcgccatggtggccaagagtagagccatcttgctgccaatggatctggatgcccacatgagcgccctgctcagcagtgggggcagctgttccgccgctgcccagcgcagcgctgccaactacaagacggccaccaggaccttccctcgggtcatccccactgccaaccagtgggactacaagaacatcattgagaagttacaggatatcatcatggccctagaagagcggctgaagcctctggtacaggctgagctctctgtccttgtggacatgctgcactggcctgagctgctttttccagagggcagtgaggcctaccagcgctgcgagagcgggggcttcctgtctaagctcatccgtcacactaagggtctcatggagtccgaggagaagttgtgcgtgaaggtgctgcggacgctgcagcagatgctgctgaagaagagcaagttcggggaccggggcaaccagctgcggaaaatgctgctgcagaattatctccagaaccgcaagtctggcgcccggggcgagctcactgaccccacgggctctggcctggatcaggactggtcagccatcgccgccacccagtgccggctggacaaggagggggccaccaagctggtgtgtgatcttatcaccagcaccaagaacgagaagatcttccaggagagcattggcctggccatccgcctgctggacgggggcaacaccgagatccagaagtctttctacaacctgatgacaagtgacaagaagtcggagcgcttcttcaaggtgcttcatgaccgcatgaagcgcgcccagcaggagaccaagtccacggtcgctgtcaacatgagcgacctgggcagccagcctcgtgaggaccgggagccggcagatcctgccaccaaaggccgcgtgtcctccttctccatgcccagctcctcccgctatttgctgggcctcggcttgcaccgagggcacgacatgagcgagcgggcgcagaacaacgagatggggacctcggtgctgatcatgcggcccatcctgcgcttcctgcagctgctgtgtgagaaccacaaccgtgacctgcaaaacttcctgcgctgtcagaacaacaagaccaactacaacctggtgtgtgagaccctgcagttcctggacatcatgtgtggcagcaccacagggggcctggggctgctggggctctacatcaacgaggacaacgtgggcctggtcatccagaccttggagaccctcacagagtactgccagggcccatgtcatgagaaccagacctgcattgtgactcacgaatccaacggcatcgacatcatcacggcgctgatcctcaatgacatcagcccgctttgcaagtaccgcatggacctggtgctgcagctcaaggacaatgcctccaagctgctcctggctctgatggagagccgacatgacagtgagaatgcagagcgcatcctcatcagccttcggcctcaggagctggtggacgtgatcaagaaggcctacctgcaggaggaggagagggagaattcagaagtgagcccacgtgaagtgggccacaacatctacatcctagcactgcagctttccaggcacaacaagcagctgcagcacctgctgaagccagtgaggcgcatccaggaggaggaggccgagggcatctcttccatgctcagcctcaacaataagcagctgtcccagatgctcaagtcctcggcccccgcccaggaggaggaagaggacccgctggcctactatgagaatcacacctcgcagattgagattgtgcggcaagaccgaagcatggagcagatcgtgttcccggtacctgccatctgccagttcctgacagaggagacgaagcaccggctcttcaccaccactgagcaggacgagcagggcagcaaagtgagcgacttcttcgaccagtcctccttcctgcacaacgagatggagtggcagcgccggcttcgcagcatgccgctcatctactggttctcccgccgcatgacgctgtggggcagcatctccttcaacctggctgtgttcatcaatatcatcattgccttcttctacccctatgtggagggcgcttccacaggcgtgctgggctcgccgctcatctcgctactcttctggatcctcatctgcttctccatcgctgcactgttcaccaagcgctacagcgtccgtcccctcatcgtggcgctgatccttcggtccatctactacctgggcattgggcccacgctcaacatcctgggtgctctcaatctgaccaacaagatcgtgtttgtggtgagcttcgtgggcaaccgtggcaccttcatccgaggctataaggccatggtcatggacatggagttcctgtaccatgtgggctacatcctgacaagcgtcctgggcctctttgcccatgagctcttctacagcatcctgctctttgacctcatctaccgagaggagacgctgtttaatgtcatcaagagtgtgacccgcaatggccgctccatcctactgaccgccctgctggccctcatccttgtgtacctcttctccatcgtgggcttcctcttcctcaaggacgacttcatcctggaggtggaccggctgcccgggaaccactccagagccagccccctggggatgccacacggagctgccacatttatgggcacatgtagtggggacaagatggactgtgtctctgaggtctcagtgcctgagatcctagaagaggacgaggagcccgatagcacggagcgggcctgtgacactctacttatgtgtatcgtgaccgtcatgaaccacggactgaggaacggtggtggcgtgggcgacatcctccgcaagccctccaaagatgagtcgctcttcccagccagagtggtgtatgaccttctgttcttctttatcgtcatcatcatcgtgttgaacctcatctttggggtgatcatcgacacctttgccgatctgcgcagcgagaagcagaagaaagaggaaatccttaagaccacgtgcttcatctgtggtctggagagggacaagtttgacaacaagacagtgtccttcgaggagcacatcaaactggagcataacatgtggaactacctgtacttcattgtactggtccgagtcaagaacaagacagactacaccggccccgagagctatgtggctcagatgatcaagaacaagaacctggactggttcccgcggatgcgtgccatgtccctggtgagcggcgagggggagggggagcagaatgagatccgcatcctgcaggagaagctcggctccaccatgaagctggtgtcgcacctcacctcccagctcaatgaactcaaggagcagatgacggagcagcggaaacggaggcagcgtctgggctttgtggatgtgcagaactgcatgagccgctgagcagagccaccgaggccccctgcaggaggtccgctgcagatggcactgcccttccttcaggtcaggtggcccaccaccctctggaagccaggtgcccaccccacctccagctctgctgcaatctccctcccccaaggcaaactggccagcaggagtgcttcacagcagtcctgcttagtaccgttgaagagactcagtttaccttagtgccttagaggatgctaggtcctctgactgacattcctcagtggtcctcacaggtggttataggggggctgcctctcacctccgagcactacattgtggctgtcccctgcccgcctgcccgccttctgtcctggatcaggaagatggtagcctgctgttagtagcttcacagggcttttatgtttaacttattcccaggggccgtcccaccctgctgtgcctgcacagttcccagtgcctgtgcctgcgcctgctggggacagtgtggccctgtcccagtagcttgatgactcagggtggaccttgctcccaggacttgtctctccccaggctttgtaggcaagcctcacaagccgagggacaggaccgggctgtgtgaaagtggcttttgacagcttctctctttttggtgtaatgttttacatgtcctgtgtcctggggactaatgtgttaattgccttaaataaattaatagactccaagtccaaaaaaaaaaaaaaaaa

SEQ ID NO: 6 - protein, 2670 aa, Mus musculus
MNEMSSFLHIGDIVSLYAEGSVNGFISTLGLVDDRCVVEPAAGDLDNPPKKFRDCLFKVCPMNRYSAQKQYWKAKQTKQDKEKIADVVLLQKLQHAAQMEQKQNDTENKKVHGDVVKYGSVIQLLHMKSNKYLTVNKRLPALLEKNAMRVTLDATGNEGSWLFIQPFWKLRSNGDNVVVGDKVILNPVNAGQPLHASNYELSDNAGCKEVNSVNCNTSWKINLFMQFRDHLEEVLKGGDVVRLFHAEQEKFLTCDEYRGKLQVFLRTTLRQSATSATSSNALWEVEVVHHDPCRGGAGHWNGLYRFKHLATGNYLAAEENPSYKGDVSDPKAAGLGAQGRTGRRNAGEKIKYRLVAVPHGNDIASLFELDPTTLQKTDSFVPRNSYVRLRHLCTNTWIQSTNAPIDVEEERPIRLMLGTCPTKEDKEAFAIVSVPVSEIRDLDFANDASSMLASAVEKLNEGFISQNDRRFVIQLLEDLVFFVSDVPNNGQNVLDIMVTKPNRERQKLMREQNILKQIFGILKAPFRDKGGEGPLVRLEELSDQKNAPYQYMFRLCYRVLRHSQEDYRKNQEHIAKQFGMMQSQIGYDILAEDTITALLHNNRKLLEKHITKTEVETFVSLVRKNREPRFLDYLSDLCVSNRIAIPVTQELICKCVLDPKNSDILIQTELRPVKEMAQSHEYLSIEYSEEEVWLTWTDRNNEHHEKSVRQLAQEARAGNAHDENVLSYYRYQLKLFARMCLDRQYLAIDEISKQLGVELLFLCMADEMLPFDLRASFCHLMLHVHVDRDPQELVTPVKFARLWTEIPTAITIKDYDSNLNASRDDKKNKFASTMEFVEDYLNNVVSEAVPFANDEKNILTFEVVSLAHNLIYFGFYSFSELLRLTRTLLGIIDCIQAPAAMLQAYEEPGGKNVRRSIQGVGHMMSTMVLSRKQSVFGASSLPAGVGVPEQLDRSKFEDNEHTVVMETKLKILEILQFILNVRLDYRISYLLSVFKKEFVEVFPMQDSGADGTAPAFDSSTATMNLDRIGEQAEAMFGVGKTSSMLEVDDEGGRMFLRVLLHLTMHDYPSLVSGALQLLFKHFSQRQEAMHTFKQVQLLISAQDVENYKVIKSELDRLRTMVEKSELWVDKKGSVKGEEVEAGATKDKKERPSDEEGFLQPHGEKSSENYQIVKGILERLNKMCGVGEQMRKKQQRLLKNMDAHKVMLDLLQIPYDKSDNKMLEILRYTHQFLQKFCAGNPGNQALLHKHLQLFLTPGLLEAETMQHIFLNNYQLCSEISEPVLQHFVHLLATHGRHVQYLDFLHTVIKAEGKYVKKCQDMIMTELTNAGDDVVVFYNDKASLAHLLDMMKAARDGVEDHSPLMYHISLVDLLAACAEGKNVYTEIKCTSLLPLEDVVTVVTHEDCITEVKMAYVNFVNHCYVDTEVEMKEIYTSNHIWTLFENFTLDMALVCNKREKRLSDPTLEKYVLTVVLDTISAFFSSPFSENSTSLQTHQTIVVQLLQSTTRLLECPWLQQQHKGSVEACVRTLAMVAKSRAILLPMDLDAHMSALLSSGGSCSAAAQRSAANYKTATRTFPRVIPTANQWDYKNIIEKLQDIIMALEERLKPLVQAELSVLVDMLHWPELLFPEGSEAYQRCESGGFLSKLIRHTKGLMESEEKLCVKVLRTLQQMLLKKSKFGDRGNQLRKMLLQNYLQNRKSGARGELTDPTGSGLDQDWSAIAATQCRLDKEGATKLVCDLITSTKNEKIFQESIGLAIRLLDGGNTEIQKSFYNLMTSDKKSERFFKVLHDRMKRAQQETKSTVAVNMSDLGSQPREDREPADPATKGRVSSFSMPSSSRYLLGLGLHRGHDMSERAQNNEMGTSVLIMRPILRFLQLLCENHNRDLQNFLRCQNNKTNYNLVCETLQFLDIMCGSTTGGLGLLGLYINEDNVGLVIQTLETLTEYCQGPCHENQTCIVTHESNGIDIITALILNDISPLCKYRMDLVLQLKDNASKLLLALMESRHDSENAERILISLRPQELVDVIKKAYLQEEERENSEVSPREVGHNIYILALQLSRHNKQLQHLLKPVRRIQEEEAEGISSMLSLNNKQLSQMLKSSAPAQEEEEDPLAYYENHTSQIEIVRQDRSMEQIVFPVPAICQFLTEETKHRLFTTTEQDEQGSKVSDFFDQSSFLHNEMEWQRRLRSMPLIYWFSRRMTLWGSISFNLAVFINIIIAFFYPYVEGASTGVLGSPLISLLFWILICFSIAALFTKRYSVRPLIVALILRSIYYLGIGPTLNILGALNLTNKIVFVVSFVGNRGTFIRGYKAMVMDMEFLYHVGYILTSVLGLFAHELFYSILLFDLIYREETLFNVIKSVTRNGRSILLTALLALILVYLFSIVGFLFLKDDFILEVDRLPGNHSRASPLGMPHGAATFMGTCSGDKMDCVSEVSVPEILEEDEEPDSTERACDTLLMCIVTVMNHGLRNGGGVGDILRKPSKDESLFPARVVYDLLFFFIVIIIVLNLIFGVIIDTFADLRSEKQKKEEILKTTCFICGLERDKFDNKTVSFEEHIKLEHNMWNYLYFIVLVRVKNKTDYTGPESYVAQMIKNKNLDWFPRMRAMSLVSGEGEGEQNEIRILQEKLGSTMKLVSHLTSQLNELKEQMTEQRKRRQRLGFVDVQNCMSR

(19) United States
(12) Patent Application Publication
(10) Pub. No.: US 2010/0035360 A1
(43) Pub. Date: Feb. 11, 2010

(54) REAGENT FOR DETECTION OF AUTOANTIBODY AND KIT FOR DIAGNOSIS OF AUTOIMMUNE DISEASE

(75) Inventor: Katsuhiko Mikoshiba, Saitama (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: Riken, Wako-shi (JP)

(21) Appl. No.: 12/306,577

(22) PCT Filed: Jun. 28, 2007
(86) PCT No.: PCT/JP07/63410

§ 371 Date: Mar. 23, 2009

(30) Foreign Application Priority Data
Jun. 29, 2006 (JP) 2006-179403

(51) Int. Cl. G01N 33/566

(52) U.S. Cl. 436/501

(57) ABSTRACT

This invention is intended to discover a novel autoantibody that can be used as a marker for an autoimmune disease and to provide an effective means for diagnosing an autoimmune disease. Disclosed is a reagent for detecting an autoantibody comprising an inositol 1,4,5-trisphosphate receptor (IP₃R) protein and/or a fragment thereof.

### TECHNICAL FIELD

The present invention relates to a reagent for detecting an autoantibody and a method for detecting an autoantibody. The present invention also relates to a diagnostic agent for an autoimmune disease.

### BACKGROUND ART

The “autoimmune disease” is a generic term referring to diseases caused by the production of antibodies against endogenous antigens by the immune system that causes excessive immune reactions to autologous normal cells or tissue. Examples of representative diseases include rheumatoid arthritis (RA), systemic lupus erythematodes (SLE), Sjogren's syndrome (SjS), systemic sclerosis (SSc), mixed connective tissue disease (MCTD), unclassified connective tissue disease (UCTD), polymyositis (PM), dermatomyositis (DM), Hashimoto's disease, and primary biliary cirrhosis (PBC). Such autoimmune diseases are diagnosed by detecting an antibody that reacts with autologous cells or tissue as an antigen (i.e., an autoantibody). Examples of such autoantibodies that have been known include anti-SS-A/Ro, anti-S-B/La, and anti-centromere antibodies. In recent years, for example, the research group to which the present inventor belongs discovered p97/VCP (Ogura, T., and Wilkinson, A. J., Genes Cells, vol. 6, pp. 575-597, 2001), which is the most abundant AAA (ATPase-associated) having a variety of cellular activities, and confirmed that this would react with the autoimmune serum obtained from a patient with primary biliary cirrhosis.

Sjogren's syndrome (SjS) is an exocrine disorder, which occurs predominantly in middle-aged women, and the ratio of men to women is 1:9. The etiology still remains unknown, and various factors, such as hereditary factors, immunological factors, environmental factors, and hormonal factors (such as decreasing estrogen level), are considered to be involved (Fox, R. I., Sjogren's syndrome, Lancet, vol. 366, pp. 321-331, 2005). The number of patients afflicted with Sjogren's syndrome in Japan is deduced to be about 300,000, and in the U.S.A. the number is deduced to be about 4,000,000.

Patients with Sjogren's syndrome (SjS) develop clinical symptoms such as dry eyes, dry mouth, and other systemic manifestation in various organs where T cells and B cells infiltrate. Further, Sjogren's syndrome is classified into primary Sjogren's syndrome (sicca alone) and secondary Sjogren's syndrome (associated with other connective tissue diseases). Serologically, 70% of patients with Sjogren's syndrome have antibodies against SS-A/Ro, and 20% to 30% of patients with primary Sjogren's syndrome (P-SjS) have antibodies against S-B/La (Miyachi, K. et al., J. Rheumatol., vol. 10, pp. 387-394, 1983). An autoantibody against SS-A/Ro recognizes a ribonucleoprotein complex composed of small single-stranded RNA (i.e., Y1 to Y5 RNA) and one or more proteins. In recent years, a 52 kDa E3 ubiquitin ligase has been reported as an autoantigen (Wada, K., and Kamitani, T., Biochem. Biophy. Res. Com., vol. 339, pp. 415-421, 2006). The SS-B antigen is considered to be an RNA polymerase III termination factor. Further, an antibody against Ki was recently identified to be a proteasome (PA 28γ), and found in less than 10% of the patients with Sjogren's syndrome (Tanahashi, N. et al., Genes Cells, vol. 2(3), pp. 195-211, 1997). Furthermore, the presence of antibodies against α-Fodrin in some patients with Sjogren's syndrome was reported (Haneji, N. et al., Science, vol. 276, pp. 604-607, 1997). Other cytoplasmic antibodies that have been found in Sjogren's syndrome include those directed against the Golgi complex (Griffith, K. J. et al., Arthritis Rheum., vol. 40, pp. 1693-1702, 1997), early endosome antigen 1 (Selack, S. et al., Clin. Immunol., vol. 109, pp. 154-164, 2003), ribosome P, mitochondria, and p97/VCP (Miyachi, K. et al., Clin. Exp. Immunol., vol. 136, pp. 568-573, 2004).

Meanwhile, an inositol 1,4,5-trisphosphate receptor (IP₃R) that is known to form a tetrameric Ca²⁺ channel in the endoplasmic reticulum is an important molecule that can regulate the calcium concentration in a living cell. This receptor is involved in neural transmission via Ca²⁺ signal transmission and has many other functions associated with morphological and biological processes in vivo. Three types of IP₃R receptors derived from three different genes have been identified in mammalians. Type 1 IP₃R (IP₃R1) is expressed mainly in brain tissue and it plays a key role in regulation of the kinetic system and the learning system (JP Patent Publication (kokai) No. 08-245698 A (1996)). It is also expressed in the smooth muscle and in endothelial cells. Two other types; i.e., type 2 IP₃R and type 3 IP₃R (IP₃R2 and IP₃R3), are expressed in various tissues and cells (Furuichi, et al., Nature, vol. 342, pp. 32-8, 1989). It has been also reported in recent years that IP₃P2 and IP₃R3 knockout mice fail to release Ca²⁺ from the endoplasmic reticulum in the cells and thus is unable to induce secretion of saliva and pancreatic juice (WO 2006/062134 and Futatsugi, A. et al., Science, vol. 309, pp. 2232-2234, 2005).

### SUMMARY OF INVENTION

The objects of the present invention are to discover a novel autoantibody that can be used as a marker for an autoimmune disease and to provide an effective means for diagnosing an autoimmune disease.

The present inventor has conducted concentrated studies in order to attain the above objects. As a result, it was found that autoantibodies against the inositol 1,4,5-trisphosphate receptor (IP₃R) are present at high frequency in the sera of patients with autoimmune diseases including Sjogren's syndrome. It was also found that sera from different autoimmune diseases may recognize different IP₃R types or domains. The present invention has been completed based on such findings.

Specifically, the present invention relates to (1) to (3) below:

(1) A reagent for detecting an autoantibody comprising an inositol 1,4,5-trisphosphate receptor (IP₃R) protein and/or a fragment thereof.

In the reagent for detecting an autoantibody, IP₃R can be a mouse or human IP₃R, for example. Also, IP₃R may be at least one selected from the group consisting of type 1 IP₃R, type 2 IP₃R, and type 3 IP₃R. Fragments of the IP₃R protein include, but not particularly limited to, a fragment comprising amino acids 224 to 604 of IP₃R1 or IP₃R2, a fragment comprising amino acids 1 to 604 of IP₃R1 or IP₃R2, a fragment comprising amino acids 1 to 2217 of IP₃R1, and a fragment comprising amino acids 1 to 2171 of IP₃R2.

In the reagent for detecting an autoantibody, the IP₃R protein and/or a fragment thereof may be immobilized on a solid phase or may be labeled.

(2) A method for detecting an autoantibody comprising detection of an anti-inositol 1,4,5-trisphosphate receptor (IP₃R) antibody in a sample.

The above method may comprise, for example, bringing a sample into contact with the IP₃R protein and/or a fragment thereof and detecting an anti-IP₃R antibody in the sample by assaying the reaction between the antibody and the IP₃R protein or a fragment thereof.

(3) A diagnostic kit for an autoimmune disease comprising the reagent for detecting an autoantibody according to (1).

An autoimmune disease to be diagnosed with the use of the aforementioned diagnostic kit may be selected from the group consisting of, for example, rheumatoid arthritis (RA), systemic lupus erythematodes (SLE), Sjogren's syndrome (SjS), systemic sclerosis (SSc), mixed connective tissue disease (MCTD), unclassified connective tissue disease (UCTD), polymyositis (PM), dermatomyositis (DM), Hashimoto's disease, primary biliary cirrhosis (PBC), ulcerative colitis, Crohn's disease, and Behcet's disease.

The reagent for detecting an autoantibody in the diagnostic kit may comprise at least one IP₃R full-length protein and/or a fragment thereof selected from the group consisting of full-length IP₃R1, full-length IP₃R2, full-length IP₃R3, and a partial fragment thereof. More preferably, the reagent may comprise at least one IP₃R protein and/or a fragment thereof selected from the group consisting of full-length IP₃R1, full-length IP₃R2, full-length IP₃R3, and a fragment comprising amino acids 224 to 604 of IP₃R1 or IP₃R2, amino acids 1 to 604 of IP₃R1 or IP₃R2, and amino acids 1 to 2217 of IP₃R1 or amino acids 1 to 2171 of IP₃R2.

The diagnostic kit may further comprise a reagent for detecting at least one autoantibody selected from the group consisting of an anti-SS-A/Ro antibody, an anti-S-B/La antibody, an anti-U1RNP antibody, an anti-Sm antibody, an anti-Scl70 antibody, an anti-Ki antibody, an anti-Ku antibody, an anti-rRNP antibody, an anti-Wa antibody, an anti-p95c/p97/VCP antibody, an anti-centromere antibody, an anti-nuclear antibody, and a rheumatoid factor.

The present invention relates to a method for evaluating possibility (risk) for and/or development of an autoimmune disease comprising detecting an anti-inositol 1,4,5-trisphosphate receptor (IP₃R) antibody in a sample obtained from a human body. More particularly, the present invention relates to a method for evaluating that a subject may become afflicted with some sort of autoimmune disease in the future or has already been afflicted therewith, when an anti-IP₃R antibody is detected in, for example, a serum sample obtained from the subject. Specifically, the present invention relates to a method involving the use of an anti-IP₃R antibody as an indicator for an autoimmune disease.

It may be obvious that whether or not a subject is afflicted with an autoimmune disease is to be finally diagnosed by a doctor based on a diagnostic method that has been already established or will be established in the future. The present invention is useful as a preliminary diagnostic method.

This description includes part or all of the disclosures in the description and/or drawings of Japanese Patent Application No. 2006-179403, based on which the present application claims priority.

### BRIEF DESCRIPTION OF DRAWINGS

FIG. 1 shows a model structure of 5 domains of IP₃R.

FIG. 2 shows the results of immunoblot analysis of three types of mouse IP₃R with serum samples from patients with various types of autoimmune diseases, serum samples from normal healthy subjects, and an anti-IP₃R antibody as a control.

FIG. 3 shows representative photographs showing the results of immunoblot analysis using serum samples from patients with RA and with SLE.

FIG. 4 shows representative photographs showing the results of immunoblot analysis using serum samples from patients with SjS.

### DESCRIPTION OF EMBODIMENTS

Hereafter, the present invention is described in detail.

The inositol 1,4,5-trisphosphate receptor (IP₃R) is known to be involved in neural transmission via Ca²⁺ signal transmission and to have many other functions associated with morphological and biological processes in vivo. The presence of antibodies against inositol 1,4,5-trisphosphate receptors (IP₃R) has been confirmed in patients with various types of autoimmune diseases. Accordingly, the present invention provides a diagnostic means and method for an autoimmune disease by detecting the presence of an autoantibody against the inositol 1,4,5-trisphosphate receptor (IP₃R) in a subject.

According to the present invention, a means that can detect the presence of an autoantibody, and more particularly, a means that can detect an autoantibody based on an antigen-antibody reaction (i.e., IP₃R protein and/or a fragment thereof), can be used to detect the presence of an autoantibody and/or to diagnosis an autoimmune disease.

IP₃R has been isolated from, for example, human, mouse, rat, Asterina, nematode, Drosophila, Xenopus, and Panulirus, and at least three IP₃R subtypes are known to exist in mammalians. However, homology among amino acid sequences of these types of IP₃R is known to be high, and differences among animal species are also known to be small (e.g., Maranto, A. R., J. Biol. Chem., 269: 1222-1230, 1994; Hattori et al., J. Biol. Chem., 279: 11967-11975, 2004; and Yamada, N. et al., Biochem. J. 302: 781-790, 1994). In the present invention, therefore, any type of IP₃R protein derived from any animal species and a fragment thereof can be used. For example, homology between human and mouse IP₃R proteins is 95% to 98%. In the examples below, a mouse IP₃R protein or a fragment thereof was actually used to detect the reaction with the serum sample from a human subject.

Representative IP₃R sequence information can be obtained from a public database. For example, the mouse type I inositol 1,4,5-trisphosphate receptor (IP₃R1) has the amino acid sequence shown in SEQ ID NO: 2 and it is encoded by the nucleotide sequence shown in SEQ ID NO: 1 (e.g., GenBank Accession Number X15373; Furuichi et al., Nature, 342: 32-38, 1989). The mouse type 2 IP₃R (IP₃R2) has the amino acid sequence shown in SEQ ID NO: 4 and it is encoded by the nucleotide sequence shown in SEQ ID NO: 3 (GenBank Accession Number AB182288; Iwai et al., J. Biol. Chem., 280: 10305-10317, 2005). The mouse type 3 IP₃R (IP₃R3) has the amino acid sequence shown in SEQ ID NO: 6 and it is encoded by the nucleotide sequence shown in SEQ ID NO: 5 (GenBank Accession Number AB182289; Iwai et al., J. Biol. Chem., 280: 10305-10317, 2005). Human IP₃R1 is registered under the GenBank Accession Numbers D26070, L38019, and U23850. Human IP₃R2 is registered under the GenBank Accession Number D26350, and human IP₃R3 is registered under the GenBank Accession Numbers D26351 and U01062. Also, JP Patent Publication (kokai) Nos. 08-245698 A (1996) and 08-134097 A (1996); Yamada et al., Biochem J. 302: 781-790, 1994; Harnick et al., J. Biol. Chem., 270: 2833-2840, 1995; Nucifora et al., Mol. Brain. Res., 32: 291-296, 1995; Yamamoto-Hino et al., Recept. Channels 2: 9-22, 1994; and Maranto, J. Biol. Chem., 269: 1222-1230, 1994 disclose information regarding such amino acid sequences and nucleotide sequences. Rat IP₃R1, IP₃R2, and IP₃R3 are registered under the GenBank Accession Numbers J05510, X61677, and L06096 (Mignery et al., J. Biol. Chem., 265: 12679-12685, 1990; Sudhof et al., Embo J., 10: 3199-3206, 1991; Blondel et al., J. Biol. Chem., 268: 11356-11363, 1993). Xenopus IP₃R1 is registered under the GenBank Accession Number D14400 (Kume et al., Cell 73: 555-570, 1993), Asterina IP₃R is registered under the GenBank Accession Number AB071372 (Iwasaki et al., J. Biol. Chem., 277: 2763-2772, 2002), Drosophila IP₃R is registered under the GenBank Accession Number D90403 (Yoshikawa et al., J. Biol. Chem. 267: 16613-16619, 1992), Panulirus IP₃R is registered under the GenBank Accession Number AF055079 (Munger et al., J. Biol. Chem. 275: 20450-20457, 2000), and nematode IP₃R is registered under the GenBank Accession Number AJ243179-82 (Baylis et al., J. Mol. Biol., 294: 467-476, 1999).

In the present invention, the IP₃R protein may be composed of an amino acid sequence derived from the amino acid sequence of the naturally occurring IP₃R protein by deletion, substitution, or addition of one or several amino acids, provided that it has reactivity with the anti-IP₃R antibody. For example, a sequence derived from the amino acid sequence shown in SEQ ID NO: 2, 4, or 6 by deletion of 1 to 5, and preferably 1 to 3, amino acids, a sequence derived from the amino acid sequence shown in SEQ ID NO: 2, 4, or 6 by addition of 1 to 5, and preferably 1 to 3, amino acids, or a sequence derived from the amino acid sequence shown in SEQ ID NO: 2, 4, or 6 by substitution of 1 to 5, and preferably 1 to 3, amino acids, with other amino acids can be used in the present invention. In particular, a sequence derived from the amino acid sequence shown in SEQ ID NO: 2, 4, or 6 by conservative substitution of one or several amino acids may be preferable. The term “conservative substitution” is known in the art, and it means that a given amino acid is substituted with an amino acid exhibiting similar properties. For example, neutral (polar) amino acids (Asn, Ser, Gln, Thr, Tyr, and Cys), neutral (non-polar, i.e., hydrophobic) amino acids (Gly, Trp, Met, Pro, Phe, Ala, Val, Leu, and Ile), acidic (polar) amino acids (Asp and Glu), or basic (polar) amino acids (Arg, His, and Lys) may be substituted with amino acids having the same properties.

In addition, as an example, a polypeptide comprising an amino acid sequence having at least 80%, preferably at least 90%, and particularly preferably at least 95% homology or identity with the amino acid sequence of the naturally-occurring IP₃R protein can also be used in the present invention. Homology or identity of amino acid sequences can be easily determined based on a method known in the art.

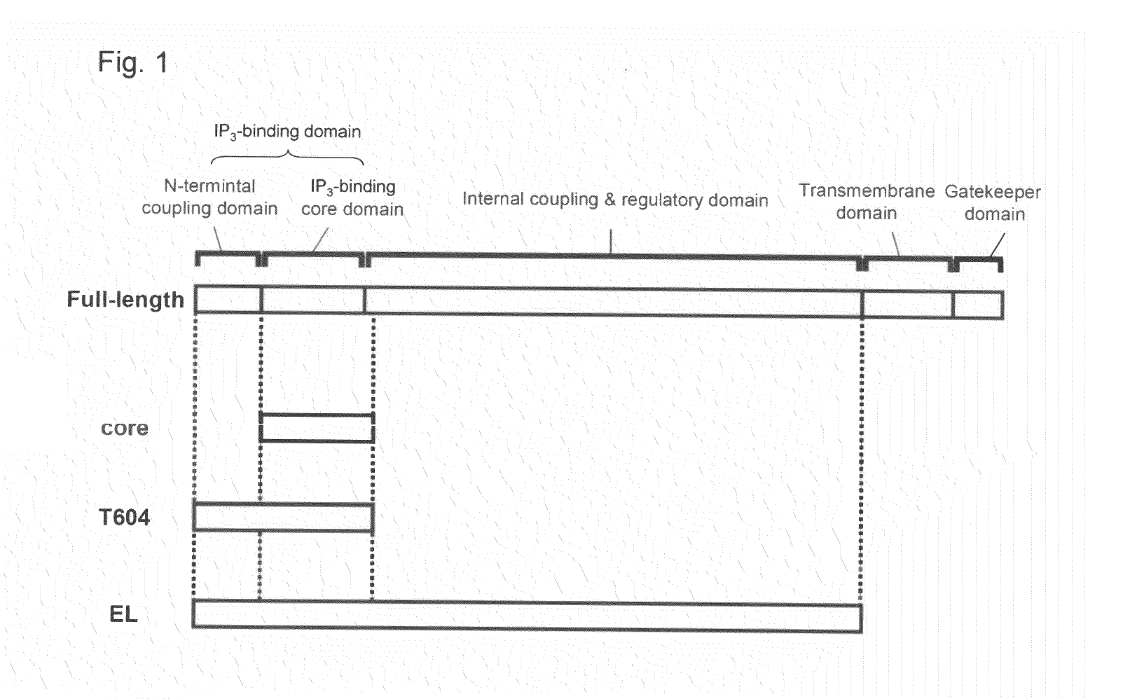

A fragment of the IP₃R protein can be a fragment of any length at any region, provided that it has reactivity with an anti-IP₃R antibody. The length of an amino acid having reactivity with an antigen (i.e., antigenicity) is known to be about 5 or 6 amino acids in the art. Accordingly, the IP₃R protein fragment can be a polypeptide comprising at least 5 or 6 amino acids. Also, IP₃R is constituted by five functional domains as shown in FIG. 1; i.e., the N-terminal coupling domain, the IP₃-binding core domain (core), the internal coupling and regulatory domain, the transmembrane domain, and the gatekeeper domain (Uchida, K. et al., J. Biol. Chem., 2003, 278: 16551-16560). A person skilled in the art could readily understand the positions of such IP₃R domains and boundaries therebetween with reference to literature or the like. The IP₃R protein fragment can be a polypeptide fragment comprising or consisting of any of the aforementioned domains, for example. Preferably, a polypeptide fragment comprising the IP₃-binding core domain (core), the IP₃-binding domain (T604) composed of the N-terminal coupling domain and the IP₃-binding core domain, or the N-terminal cytoplasmic region (EL) composed of the N-terminal coupling domain, the IP₃-binding core domain and the internal coupling and regulatory domain is used (FIG. 1). The core (IP₃-binding core domain) is located at amino acid residues 224 to 604 of mouse IP₃R1 and IP₃R2, T604 (IP₃-binding domain) is located at amino acid residues 1 to 604 of mouse IP₃R1 and IP₃R2, and EL (the N-terminal cytoplasmic region) is located at amino acid residues 1 to 2217 of mouse IP₃R1 or amino acid residues 1 to 2171 of IP₃R2, although locations are not limited thereto. A reference may be made to, for example, Uchida et al. (supra) or JP Patent Publication (kokai) Nos. 2005-304360 A, 2000-135095 A, and 2005-58116 A regarding examples of such fragments.

The IP₃R protein or a fragment thereof may be naturally isolated, or generated by chemical synthesis or recombinant technique based on sequence information.

When the IP₃R protein is naturally isolated, known techniques for isolation and purification can be used. For example, the IP₃R protein can be easily purified via affinity chromatography using an antibody against the IP₃R protein (see JP Patent Publication (kokai) No. 06-135997 A (1994)).

When a gene recombinant technique is used, a nucleic acid encoding the IP₃R protein or a fragment thereof can be obtained by reverse transcription polymerase chain reaction (RT-PCR) using primers designed based on the IP₃R gene sequence with the use of mRNA purified from RNA extracted from biological tissue or cultured cells, or by screening of the cDNA library using probes designed based on the IP₃R gene sequence. Alternatively, a nucleic acid encoding the IP₃R protein or a fragment thereof can be obtained by extracting DNA from biological tissue or cultured cells and performing nucleic acid amplification using the DNA as a template and primers designed based on IP₃R gene sequences (e.g., PCR). Methods for preparing a nucleic acid encoding the mutated IP₃R protein or a fragment thereof are known in the art.

In the present invention, an expression vector for expressing the recombinant IP₃R protein or a fragment thereof can be obtained by ligating the above nucleic acid to an adequate vector. A transformant can be prepared by introducing the nucleic acid or expression vector into a host cell so that the target protein can be expressed.

Any conventional vector, such as a plasmid vector, a phagemid vector, a virus vector, or an artificial chromosome, can be used. Plasmid DNA includes bacteria-derived plasmid (e.g., pBluescript) and yeast-derived plasmid. Phagemid DNA includes λ phage (e.g., λgt10 or λZAP). Further, transformants can be prepared using animal virus vectors such as retrovirus, adenovirus, or vaccinia virus vectors, insect virus vectors such as baculovirus vectors (e.g., pBlueBac4.5 and pFastBac1), bacterial artificial chromosomes (BACs), yeast artificial chromosomes (YACs), or human artificial chromosomes (HACs).

A nucleic acid can be inserted into a vector by, for example, cleaving the purified nucleic acid with adequate restriction enzymes and inserting the resultant into the restriction enzyme site or multicloning site of vector DNA to ligate the nucleic acid to the vector. A vector should be constructed so that it can be autonomously replicated in the host cell or a nucleic acid on the vector may be incorporated into the genome of the host cell to express the IP₃R protein or a fragment thereof in the host cell. So, a promoter and a nucleic acid as well as, if necessary, a cis-element such as an enhancer, a splicing signal, a poly A addition signal, a selection marker, a ribosome binding (SD) sequence, a homologous sequence, or the like may be preferably ligated to the vector. Examples of selection markers include dihydrofolate reductase genes, ampicillin resistant genes, and neomycin resistant genes. In order to facilitate purification of the IP₃R protein or a fragment thereof, a signal sequence, an His tag, or the like may be added. These sequences are ligated to the vector using known DNA ligase. The sequences are annealed and then ligated to the vector to prepare expression vectors.

Host cells to be used for transformation are not particularly limited, provided that they are capable of expressing the introduced nucleic acid and producing proteins. Examples thereof include bacteria (e.g., Escherichia coli BL21), yeast (e.g., Saccharomyces cerevisae), animal cells (e.g., COS cells and CHO cells), and insect cells (e.g., Sf9 cells and Sf21 cells).

A method for introducing a nucleic acid or expression vector into bacteria or yeast is not particularly limited, provided that it allows DNA be introduced into them. Examples thereof include electroporation, the spheroplast method, and the lithium acetate method. Examples of methods for introducing a nucleic acid or expression vector into an animal cell or insect cell include electroporation, the calcium phosphate method, and lipofection.

A transformant is selected based on properties of the marker gene constructed in the gene to be introduced. When a neomycin resistant gene is used, for example, a cell exhibiting resistance to the G418 drug is selected.

The IP₃R protein or a fragment thereof can be obtained by culturing the transformant into which a nucleic acid encoding the IP₃R protein or a fragment thereof has been introduced and collecting the IP₃R protein or a fragment thereof from the culture. The term “culture” refers to any of a culture supernatant, a cultured cell, or a disrupted cell. A method involving culture of a transformant in a medium is carried out in accordance with a conventional technique used for host cell culture.

A medium for culturing a transformant obtained with the use of a bacterial or yeast host may be natural or synthetic medium, provided that such medium comprises a carbon source, a nitrogen source, an inorganic salt, and the like and it can efficiently culture the transformant. Culture is generally carried out under aerobic conditions, such as agitation culture or aeration agitation culture conditions at approximately 20° C. to 40° C. for approximately 1 to 24 hours. During culture, the pH level is maintained at a roughly neutral level. During culture, antibiotics, such as ampicillin or tetracycline, may be added to the medium according to need. As a medium for culturing a transformant obtained with the use of an animal or insect host cell, for example, conventional RPMI 1640 medium, DMEM medium, or these medium supplemented with fetal calf serum or the like may be used. In general, culture is conducted in the presence of 5% CO₂ at approximately 37° C. for approximately 1 to 7 days. During culture, antibiotics, such as streptomycin or penicillin, may be added to the medium according to need.

When the IP₃R protein or a fragment thereof is secreted within the cells or bacteria, the cells or bacteria may be disrupted to extract proteins after the culture. When the IP₃R protein or a fragment thereof is secreted outside the cells or bacteria, the culture solution may be used in that state, or cells or bacteria may be removed via centrifugation or other means.

The IP₃R protein or a fragment thereof produced by chemical synthesis or recombinant technique can be isolated and purified by a general biochemical technique used for protein isolation/purification, such as ammonium sulfate precipitation, gel chromatography, ion-exchange chromatography, or affinity chromatography, and such technique may be used alone or in adequate combination.

Whether or not the target IP₃R protein or a fragment thereof was obtained can be confirmed by polyacrylamide gel electrophoresis, sodium dodecyl sulfate-polyacrylamide gel electrophoresis (SDS-PAGE), or other means.

The IP₃R protein fragment is chemically synthesized in accordance with a known technique for peptide synthesis with the use of, for example, a commercially available peptide synthesizer or peptide synthesis kit. Techniques for peptide synthesis are described in literature such as Peptide Synthesis, Interscience, New York, 1996 or The Proteins, vol. 2, Academic Press Inc., New York, 1976.

Alternatively, the IP₃R protein fragment can be obtained by chemically or enzymatically cleaving the IP₃R protein, which has been isolated or prepared by recombinant technique, as described above.

Whether or not the resulting IP₃R protein or a fragment thereof can react with an autoantibody can be confirmed by allowing the protein or a fragment thereof to react with the serum from a patient with an autoimmune disease, or with the known anti-IP₃R antibody.

The IP₃R protein or a fragment thereof can be used to detect an anti-IP₃R antibody in a sample. Such detection can be carried out by any method, provided that such method involves assay of an antigen-antibody reaction; i.e., an immunoassay method. For example, an anti-IP₃R antibody can be detected via immunoassay techniques, such as enzyme immunoassay (e.g., ELISA or EIA), fluorescent immunoassay, radioimmunoassay (RIA), immunochromatic assay, and immunoblot assay, the Octarony method (double immune diffusion method), immunohistochemical staining, or immune electron microscopy. The test samples are not particularly limited, provided that the presence of an autoantibody therein is to be detected. Samples that can be used include blood samples such as whole blood, serum, or plasma, body fluid samples such as saliva, spinal fluid, synovial fluid, or urine, and solid samples such as cells or tissue. A blood sample from a subject in which the presence of an autoantibody is to be detected is particularly preferable.

An immunoassay technique comprises allowing anti-IP₃R antibodies in a sample to bind to the IP₃R protein or a fragment thereof and detecting the binding between the antibodies and the IP₃R protein or a fragment thereof to thereby detect anti-IP₃R antibodies. In the present invention, the term “detection” refers not only to detection of the presence or absence of anti-IP₃R antibodies but to quantitative detection of anti-IP₃R antibodies.

Typically, immunoassay of anti-IP₃R antibodies comprises bringing the test samples into contact with the IP₃R proteins or fragments thereof and detecting the IP₃R proteins or fragments thereof bound to the anti-IP₃R antibodies via a technique known in the art. The term “contact” refers to conditions under which the anti-IP₃R antibodies in the sample would be accessible to the IP₃R proteins or fragments thereof, so that the anti-IP₃R antibodies could bind to the IP₃R proteins or fragments thereof. For example, such contact can be achieved by mixing of a liquid sample with a solution containing the IP₃R proteins or fragments thereof, adding the IP₃R proteins or fragments thereof to the liquid sample, applying the liquid sample to the pores or wells of a gel plate containing the IP₃R proteins or fragments thereof, or coating the solid sample with a solution containing the IP₃R proteins or fragments thereof.

A liquid-phase or solid-phase immunoassay may be employed. From the viewpoint of the ease of detection, use of a solid-phase system is preferable. The format of immunoassay is not limited, and it may be carried out in the format of sandwich assay or competitive assay, as well as in the form of direct solid-phase assay.

Assay can be carried out in accordance with a known technique (Ausubel, F. M., et al. (ed.), Short Protocols in Molecular Biology, Chapter 11, “Immunology,” John Wiley & Sons, Inc., 1995). For example, immunoblotting (Western blotting) can be employed. Alternatively, a complex of the IP₃R protein or a fragment thereof and an antibody may be separated via a known technique (e.g., chromatography, salting-out, alcohol precipitation, an enzyme method, a solid-phase method, or immunodiffusion), and the label signal may then be detected.

When a solid-phase immunoassay is employed, for example, the IP₃R proteins or fragments thereof may be immobilized on a solid-phase support or carrier (e.g., resin, membrane, film, bead, or gel), or samples may be immobilized. For example, the IP₃R proteins or fragments thereof are immobilized on a solid-phase support, the support is washed with an adequate buffer, and the support is then treated with the samples. Subsequently, a solid-phase support is washed with a buffer to remove unbound samples. The amount of the bound antibodies on the solid-phase support is then detected by a conventional means, and binding of autoantibodies to the IP₃R proteins or fragments thereof in the samples can be detected.

Examples of a solid-phase support or carrier include a synthetic organic polymer compound (e.g., polyvinyl chloride, polystyrene, polyvinyl alcohol, polyacrylamide, or polypropylene), a polysaccharide (e.g., a dextran derivative, cellulose, or agarose gel), and an inorganic polymer compound (e.g., glass, silica, or silicon). A carrier may be in any form, such as a plate, particle, tube, fiber, membrane, or microparticle. The IP₃R proteins or fragments thereof may be bound to a carrier via physical adsorption, ionic binding, covalent binding, or other means. Alternatively, they may be bound via other groups (i.e., linkers).

A binding activity of an antibody can be assayed in accordance with a well-known technique. A person skilled in the art can determine an effective and optimal assay technique based on the type and the format of immunoassay to be employed, the type and the target of a label to be used, and other factors.

In an embodiment of the present invention, the reaction of autoantibodies in the sample with the IP₃R proteins or fragments thereof can be easily detected directly by labeling the IP₃R proteins or fragments thereof or indirectly by using a labeled secondary antibody, a biotin-avidin complex, or the like. Examples of labels that can be used in the present invention and methods for detecting the same are described below.

In the case of enzyme immunoassay, for example, peroxidase, β-galactosidase, alkaline phosphatase, glucose oxidase, acetylcholine esterase, lactate dehydrogenase, or amylase can be used. Also, an enzyme inhibitor, a coenzyme, or the like can be used. IP₃R proteins or fragments thereof can be bound to such enzymes by a conventional technique involving the use of a crosslinking agent, such as a glutaraldehyde or maleimide compound.

In the case of fluorescent immunoassay, for example, fluorescein isothiocyanate (FITC) or tetramethylrhodamine isothiocyanate (TRITC) can be used. Such fluorescent labels can be bound in accordance with a conventional technique.

In the case of radioimmunoassay, for example, tritium, iodine¹²⁵, or iodine¹³¹ can be used. Radioactive labels can be bound by a known technique, such as the chloramine-T method or the Bolton-Hunter method.

When the IP₃R protein or a fragment thereof is directly labeled with a label as described above, for example, the sample is brought into contact with the labeled IP₃R protein or a fragment thereof to form a complex of the IP₃R protein or a fragment thereof and an autoantibody. The unbound and labeled IP₃R protein or a fragment thereof is then separated, and the amount of autoantibodies in the sample can then be assayed based on the amount of the bound or unbound and labeled IP₃R protein or a fragment thereof.

When the labeled secondary antibody is used, for example, the IP₃R protein or a fragment thereof is allowed to react with the sample (the primary reaction), and the labeled secondary antibody is further allowed to react with the resulting complex (the secondary reaction). The primary reaction and the secondary reaction may be carried out in the opposite order, simultaneously or at different times. As a result of the primary reaction and the secondary reaction, the complex of the IP₃R protein or a fragment thereof, the autoantibody, and the labeled secondary antibody, or the complex of the autoantibody, the IP₃R protein or a fragment thereof, and the labeled secondary antibody is formed. Further, the unbound labeled secondary antibody is separated, and the amount of autoantibodies in the sample can then be determined based on the amount of the bound or unbound labeled secondary antibodies.

When a biotin-avidin complex is used, the biotinylated IP₃R protein or a fragment thereof is allowed to react with a sample. Alternatively, the IP₃R protein or a fragment thereof is allowed to react with a sample, the biotinylated secondary antibody is allowed to react therewith. Then, the resulting complex is allowed to react with labeled avidin. Since avidin can bind specifically to biotin, a signal of the label added to avidin may be detected so as to assay the binding between an autoantibody and the IP₃R protein or a fragment thereof. A label to be added to avidin is not particularly limited and preferably includes, for example, an enzyme label, such as peroxidase or alkaline phosphatase.

The label signal can also be detected in accordance with a technique known in the art. When an enzyme label is used, for example, a substrate that is degraded via an enzyme reaction and develops color may be added. The amount of the substrate degraded may be optically assayed to determine enzyme activity, and the determined enzyme activity may be converted so as to correspond to the amount of the bound autoantibodies. Alternatively, the dilution series for the sample may be used to evaluate the abundance of autoantibodies based on the dilution ratio. A substrate is selected in accordance with the type of enzyme to be used. When peroxidase is used as an enzyme, for example, 3,3′,5,5′-tetramethylbenzidine (TMB) or diaminobenzidine (DAB) may be used. When alkaline phosphatase is used as an enzyme, for example, paranitrophenol may be used. A fluorescent label can be detected and quantified with the use of, for example, a fluorescent microscope or a plate reader. When a radioactive label is used, the dose of radiation emitted by the radioactive label may be quantitated using a scintillation counter or the like.

In a preferable embodiment of the present invention, a plurality of proteins selected from the group consisting of at least one type of IP₃R protein or a fragment thereof (preferably a type 1, type 2, and type 3 IP₃R protein or an IP₃R protein fragment (e.g., core, T604, or EL)) are allowed to bind to a solid phase (e.g., a membrane, chip, or plate) and then subjected to blocking treatment. The blood sample obtained from the subject is then applied to the solid phase. Preferably, the dilution series for the blood sample is prepared and then applied to the solid phase. After the solid phase is washed and the unbound sample is removed, the labeled secondary antibody (e.g., an anti-human IgG antibody) is then applied thereto. After the unreacted secondary antibody is removed by washing, an autoantibody in the sample is detected based on a label on the solid phase.

As described above, use of the reagent for detecting an autoantibody of the present invention can facilitate and simplify the detection of anti-IP₃R antibodies in a sample.

Also, the reagent for detecting an autoantibody of the present invention can be used for a diagnostic kit for an autoimmune disease. An autoimmune disease to be diagnosed is not particularly limited, provided that such disease involves expression of an anti-IP₃R antibody as the autoantibody. Examples of such disease include rheumatoid arthritis (RA), systemic lupus erythematodes (SLE), Sjogren's syndrome (SjS), systemic sclerosis (SSc), mixed connective tissue disease (MCTD), unclassified connective tissue disease (UCTD), polymyositis (PM), dermatomyositis (DM), Hashimoto's disease, primary biliary cirrhosis (PBC), ulcerative colitis, Crohn's disease, and Behcet's disease.

The diagnostic kit of the present invention comprises the above reagent for detecting an autoantibody, i.e., the IP₃R protein and/or a fragment thereof. The diagnostic kit may comprise a single type of IP₃R protein and/or a fragment thereof or a plurality of types of IP₃R proteins and/or fragments thereof. Preferably, the kit comprises at least one IP₃R protein and/or a fragment thereof selected from the group consisting of full-length IP₃R1, full-length IP₃R2, full-length IP₃R3, and a fragment comprising amino acids 224 to 604 of IP₃R1 or IP₃R2, amino acids 1 to 604 of IP₃R1 or IP₃R2, and amino acids 1 to 2217 of IP₃R1 or amino acids 1 to 2171 of IP₃R2. Particularly preferably, the kit comprises all the full-length IP₃R proteins and fragments thereof mentioned above.

The reagent for detecting an autoantibody included in the diagnostic kit may be immobilized on a solid phase as described above. For example, the type of IP₃R protein or fragment thereof may be immobilized on a solid phase, or a plurality of types of IP₃R proteins and/or fragments thereof may be immobilized on the same or a different solid phase. Further, the reagent for detecting an autoantibody may be labeled as described above.

The diagnostic kit of the present invention may further comprise other components that are useful for carrying out an immunoassay. For example, the kit can comprise components that are necessary for detecting an antigen-antibody reaction, such as by immunoprecipitation, immunoassay (e.g., EIA, RIA, or ELISA), or immunoblotting. Examples thereof include a buffer, a reagent for sample treatment, a label, a secondary antibody, a positive control, and a negative control.

The form of the diagnostic kit is not particularly limited, and the kit can be in the form of a container containing a solution containing a reagent for detecting an autoantibody, a solid phase (e.g., a membrane, chip, or plate) on which the reagent for detecting an autoantibody has been immobilized, or a container containing the lyophilized reagent for detecting an autoantibody.

The diagnostic kit can detect an anti-IP₃R antibody contained in a sample from a subject who is to be diagnosed regarding an affliction involving an autoimmune disease or a patient having an autoimmune disease. Thus, whether or not the subject is afflicted with an autoimmune disease, the condition and the progress of the disease, and the risk of a patient being afflicted with the disease can be rapidly and simply determined. Such diagnostic kits functioning via immunoassay are well-known, and a person skilled in the art can use the diagnostic kit of the present invention in accordance with a known immunoassay technique.

The diagnostic kit of the present invention may comprise other components that are used for detecting an autoantibody and/or for diagnosing an autoimmune disease. Examples of such components include reagents for detecting autoantibodies, such as anti-SS-A/Ro antibodies, anti-S-B/La antibodies, anti-U1RNP antibodies, anti-Sm antibodies, anti-Scl70 antibodies, anti-Ki antibodies, anti-Ku antibodies, anti-rRNP antibodies, anti-Wa antibodies, anti-p95c/p97/VCP antibodies, anti-centromere antibodies (ACA), anti-nuclear antibodies (ANA), and rheumatoid factor (RF). A reagent that is commercially available from, for example, Medical & Biological Laboratories Co., Ltd. (MBL) may be used. Alternatively, reagents disclosed in the relevant literature may be used.

In the diagnostic kit, the other components for detecting an autoantibody and/or for diagnosing an autoimmune disease may be immobilized on a solid phase. In such a case, the other components may be immobilized separately. Alternatively, such components may be immobilized on a solid phase with the reagent for detecting an autoantibody (i.e., the IP₃R protein and/or a fragment thereof) of the present invention. For example, all components are preferably immobilized on the same solid phase and the presence of various autoantibodies in the sample can be detected by a single reaction procedure.

Detection of the presence of an anti-IP₃R antibody in a sample or the presence of an anti-IP₃R antibody and another autoantibody in the sample enables simple and highly accurate diagnosis of an autoimmune disease.

Hereafter, the present invention is described in greater detail with reference to the following examples, although the technical scope of the present invention is not limited thereto.

### EXAMPLES

### Example 1

In the examples, the presence of anti-IP₃R antibodies in patients with various types of autoimmune diseases was examined.

Patients included 74 patients with Sjogren's syndrome (35 patients with primary SjS (P-SjS)) and 39 patients with secondary SjS (S-SjS)), 144 patients with rheumatoid arthritis (RA), 96 patients with other connective tissue diseases (CTP), and 33 normal healthy subjects (NHS). Sjogren's syndrome (SjS) was diagnosed in accordance with the European or international criteria for SjS (Vitali, C. et al., Ann. Rheum. Dis., 53: 637-647, 1994; Vitali, C., Ann. Rheum. Dis., 62: 94-95, 2003; author's reply 95). When the subject satisfied the conditions of two of the following four items, the subject was diagnosed as having SjS: (1) an histopathological examination showing more than 50 lymphocytes infiltrated around each labial small ducts or lacrimal small ducts; (2) an oral examination of stage 1 or higher by sialography (Rubin, P. and Holt, J. F., Am. J. Roentgenol., 1957; 77: 575-598) or low secretion of saliva (i.e., less than 10 ml per 10 min. by Gum test or less than 2 g per 2 min. by Saxon test) and secretion dysfunction by salivary scintigraphy; (3) an ophthalmic examination showing 5-mm wet per 5 minutes by Schirmer's test and score of 3 or higher by van Bijsterveld in the Rose Bengal test; and (4) a serological examination showing the presence of anti-SS-A/Ro or anti-S-B/La antibodies (Fujibayashi, K. et al., The report to the Japanese Ministry of Welfare, 135-138, 1999).

RA, systemic lupus erythematodes (SLE), and systemic sclerosis (SSc) were diagnosed in accordance with standard criteria. Written consents were obtained from all patients and doctors.

Immunoblotting was carried out in the following manner in order to detect anti-IP₃R antibodies. As described in Iwai et al. (Iwai, M. et al., J. Biol. Chem., 2005, 18: 280: 10305-17), microsome fractions were prepared from Sf9 cells that overexpress mouse IP₃R. Specifically, Sf9 cells were infected with recombinant baculoviruses containing cDNA encoding a type of mouse IP₃R protein to express various types of IP₃R proteins in Sf9 cells. The IP₃R proteins were separated on 5% SDS-PAGE and then transferred to a polyvinylidene difluoride (PVDF) membrane. The membrane was blocked with 3% skim milk in PBST (PBS+0.05% Tween 20) and then incubated with blood serum. Incubation with the serum (1:300) was carried out at 4° C. overnight. The membrane was washed three times with PBST and then incubated with an anti-human IgG (h&l) antibodies-horseradish peroxidase complex (BETHYL laboratories, INC; 1:2000) at room temperature for 1 hour. With the use of an ECL Western blotting detection reagent (GE Healthcare), the blot was developed. The signal intensity was calculated using Scion Image software (Scion Corporation). The obtained value was designated as an average of at least three measurements. Such signal intensity was semiquantitative, but serum exhibiting signal intensity of 80 or more relative to any type of IP₃R protein (at least twice the average of NHS) was designated as a positive control. In the examples, numerical values of two groups were compared via a χ square test, and the numeral values were determined to be significant when the p value was less than 0.05.

The results are shown in Table 1 below. The anti-IP₃R antibodies were observed in 22 of 35 P-SjS patients (62.9%) and 12 of 39 S-SjS patients (30.8%). The frequency of the anti-IP₃R antibodies observed in these SjS patients was significantly higher than the frequency (9.1%) observed in normal healthy subjects. Also, anti-IP₃R antibodies were observed in 53.3% of patients with RA (66 of 124 patients), 48.2% of patients with other CTD diseases (26 of 54 patients), and 39.2% of patients with other autoimmune diseases (9 of 23 patients).

TABLE 1
Incidence of autoantibodies against IP₃R
in Sjogren's syndrome and other disease conditions

| | Number of | Number of | Percentage of |
|---|---|---|---|
| | positive | subjects | positive |
| Disease | subjects | tested | subjects (%) |
| Sjogren's syndrome (SjS) | 34 | 74 | 45.8 |
| Primary (p)-SjS | 22 | 35 | 62.9 |
| Secondary (s)-SjS | 12 | 39 | 30.8 |
| Rheumatoid arthritis (RA) | 66 | 124 | 53.3 |
| Other CTD | 26 | 54 | 48.2 |
| SLE | 13 | 26 | 50.0 |
| SSc | 9 | 17 | 52.4 |
| MCTD | 1 | 4 | 25.0 |
| UCTD | 2 | 5 | 40.0 |
| DM/PM | 1 | 2 | 50.0 |
| Other autoimmune disease | 9 | 23 | 39.2 |
| PBC | 4 | 6 | 66.7 |
| Hashimoto's disease | 2 | 7 | 28.6 |
| Others | 3 | 10 | 30.0 |
| NHS | 3 | 33 | 9.1 |

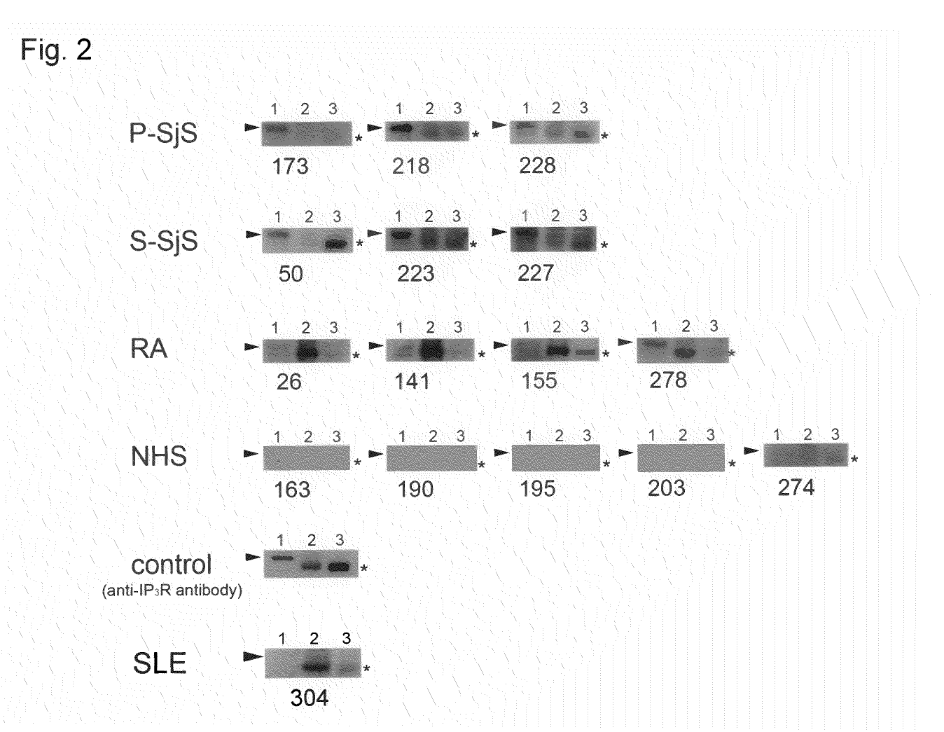

FIG. 2 shows different patterns (1 set) obtained by immunoblotting regarding the reactivity of recombinant proteins IP₃R1, IP₃R2, and IP₃R3 with representative serum samples from P-SjS patients, S-SjS patients, RA patients, and SLE patients, serum samples from normal healthy subjects (NHS), and rabbit anti-IP₃R antibody (Hattori, M. et al., J. Biol. Chem., 2004, 279, 11967-75). In FIG. 2, the numbers shown above the photographs (1 to 3) represent IP₃R types, the position of IP₃R1 is indicated by arrowheads, and the positions of IP₃R2 and IP₃R3 are indicated by asterisks. The numbers shown below the photographs represent the serum sample numbers from subjects.

Three serum samples obtained from P-SjS patients (Nos. 173, 218, and 228) produced a stronger band in type 1 than that in type 2 or 3. Three serum samples obtained from S-SjS patients (Nos. 50, 223, and 227) yielded a stronger band in type 3 than that in type 1 or 2. Four serum samples obtained from RA patients (Nos. 26, 141, 155, and 278) produced a stronger band in type 2 than that in type 1 or 3. Four serum samples obtained from NHS yielded no significant band, and rabbit positive control serum samples reacted specifically to IP₃R1, IP₃R2, and IP₃R3. Interestingly, autoantibodies reacting with the IP₃R2 dominant were found in 19 of 124 (15.4%) RA patients having no sicca symptoms.

This suggests that anti-IP₃R antibodies in the serum samples obtained from patients with autoimmune diseases would recognize different epitopes depending on disease type.

### Example 2

In this example, the presence of anti-IP₃R antibodies in patients with various types of autoimmune diseases and the presence of other known autoantibodies were examined.

Specifically, the presence of autoantibodies (including SS-A/Ro antibodies, S-B/La antibodies, U1RNP antibodies, Sm antibodies, Scl70 antibodies, Ki antibodies, Ku antibodies, rRNP antibodies, Wa antibodies, and p95c/p97/VCP antibodies) in patients with autoimmune disease in Example 1 was screened for by double immunodiffusion (Miyachi, K., Matsushima, H., Hankins, R. W. et al., A novel antibody directed against a three-dimensional configuration of a 95-kDa protein in patients with autoimmune hepatic diseases. Scand. J. Immunol., 1998; 136: 568-573). In order to determine accurate frequency, 39 S-SjS patients were reclassified to CTD. The resulting numbers for RA, SLE, SSc, and MCTD were 144, 34, 26, and 6 patients, respectively.

Further, antibodies against SS-A/Ro, S-B/La, U1RNP, Sm, and Scl70 (topoisomerase 1) were confirmed by ELISA using commercially available kits. Anti-Ki antibodies, anti-Ku antibodies (Mimori, T. et al., Proc. Natl. Acad. Sci., U.S.A., 1990; 87: 1777-81), anti-rRNP antibodies, anti-Wa antibodies (Miyachi, K. et al., J. Rheumatol., 1991; 18: 373-378), anti-WS antibodies (Matsumura, M. et al., Arthritis Rheum., 1996; 44: 877-882), and anti-p97/VCP antibodies (Miyachi, K. et al., Clin. Exp. Immunol., 2004; 136: 568-73) were confirmed by immunoprecipitation. Anti-centromere antibodies were confirmed by known RIA or ELISA techniques (Kashiwazaki et al., Rinshou meneki (clinical immunology) 21, suppl. 14, Spring Special Edition: 571-578, 1989).

The results are shown in Table 2. The frequencies of anti-SS-A/Ro antibodies found in patients with P-SjS and S-SjS were 22 of 35 patients (62.9%) and 23 of 39 patients (59%), respectively. The anti-centromere antibodies were found in 3 of 35 (8.6%) patients with P-SjS, 4 of 39 (10.3%) patients with S-SjS, and 10 of 26 (38.5%) patients with SSc. The anti-Ki antibodies were found in 1 of 35 (2.9%) patients with P-SjS, 4 of 39 (10.3%) patients with S-SjS, and 8 of 34 (23.6%) patients with SLE.

TABLE 2
Frequencies of various autoantibodies found in cases of Sjogren's syndrome and other autoimmune diseases

| | P-SjS | S-SjS | RA | SLE | SSc | MCTD |
|---|---|---|---|---|---|---|
| Number of subjects | 35 (2:33) | 39 (1:38) | 144 (26:118) | 34 (1:33) | 26 (1:25) | 6 (0:6) |
| (male:female) | | | | | | |
| Mean age | 62 (22 to 92) | 59 (22 to 84) | 62 (21 to 87) | 51 (23 to 81) | 61 (52 to 85) | 49 (37 to 58) |
| Anti-IP₃R | 22 (62.9%) | 12 (30.8%) | 70 (48.7%) | 16 (47.1%) | 9 (34.7%) | 1 (16.7%) |
| Anti-SS-A/Ro | 22 (62.9%) | 23 (59.0%) | 12 (8.4%) | 16 (47.1%) | 4 (15.4%) | 3 (50%) |
| Anti-SS-B/La | 4 (11.5%) | 0 | 0 | 0 | 0 | 0 |
| Anti-centromere | 3 (8.6%) | 4 (10.3%) | 1 (0.7%) | 2 (5.9%) | 10 (38.5%) | 0 |
| Anti-U1RNP | 0 | 5 (12.9%) | 0 | 6 (17.7%) | 1 (3.9%) | 6 (100%) |
| Anti-Ki | 1 (2.9%) | 4 (10.3%) | 0 | 8 (23.6%) | 0 | 0 |
| Anti-Topol | 0 | 0 | 0 | 0 | 4 (15.4%) | 0 |

Also, among patients with Sjogren's syndrome, it was examined whether or not anti-SS-A/Ro antibody-negative but anti-IP₃R antibody-positive patients were included. The results are shown in Table 3. Anti-SS-A/Ro antibodies were found in 22 of 35 (62.9%) patients with primary Sjogren's syndrome (P-SjS) and in 23 of 39 (59.0%) patients with secondary Sjogren's syndrome (S-SiS). Among 29 patients having no anti-SS-A/Ro antibodies, anti-IP₃R antibodies were found in 7 of 13 patients with P-SjS and 5 of 16 patients with S-SjS. Accordingly, anti-IP₃R antibody was positive in 12 of 29 (41.4%) anti-SS-A/Ro antibody-negative patients with Sjogren's syndrome (Table 3).

TABLE 3
Frequency of anti-IP₃R antibodies in anti-SS-A/Ro antibody-negative
patients with Sjogren's syndrome

| | Number of | | | Anti-SS-A(−) |
|---|---|---|---|---|
| | SjS cases | Anti-SS-A(+) | Anti-SS-A(−) | Anti-IP₃R(+) |
| P-SjS | 35 | 22 | 13 | 7/13 |
| S-SjS | 39 | 23 | 16 | 5/16 |
| Total | 74 | 45 | 29 | 12/29 |
| | | | | (41.4%) |

Accordingly, detection of anti-IP₃R antibodies can be useful for diagnosis of anti-SS-A/Ro-negative Sjogren's syndrome, and detection of anti-IP₃R antibodies in combination with detection of anti-SS-A/Ro antibodies or other autoantibodies enable highly accurate diagnosis of various types of autoimmune diseases.

### Example 3

In this example, the region of IP₃R recognized by the serum samples from patients with autoimmune diseases was examined.

At the outset, recombinant proteins of the IP₃-binding core domain (core), the IP₃-binding domain (T604), and the N-terminal cytoplasmic region (EL) of mouse IP₃R were prepared as antigens. Specifically, the IP₃-binding core domain (core; amino acids 224 to 604 of SEQ ID NO: 2 or 4) and the IP₃-binding domain (T604; amino acids 1 to 604 of SEQ ID NO: 2 or 4) of mouse IP₃R were expressed in Escherichia coli BL21 codonplus (Stratagene) and then purified on HiTrap heparin HP column (GE Healthcare) (Iwai M in preparation). cDNA encoding the N-terminal cytoplasmic region of mouse IP₃R1 (ELₘ₁; amino acids 1 to 2217 of SEQ ID NO: 2) was inserted into pBlueBac4.5 baculovirus transfer vector. cDNA encoding the N-terminal region of mouse IP₃R2 (ELₘ₂; amino acids 1 to 2171 of SEQ ID NO: 4) was inserted into pFastBac1 baculovirus transfer vector. The recombinant baculovirus carrying ELₘ₁ was prepared using the Bac-N-Blue™ Transfection Kit (Invitrogen). The recombinant baculovirus carrying ELₘ₂ was prepared using the Bac-to-Bac Baculovirus Expression System (Invitrogen). The recombinant viruses were amplified in Sf9 cells and used for expression. The Sf9 cells were cultured and transfected as described in Ando et al. (Ando, H. et al., J. Biol. Chem., 2003, 278: 10602-12). Soluble fractions containing recombinant proteins were prepared as described in Ando et al. (supra). The proteins were separated on 7.5% SDS-PAGE, and immunoblotting was carried out in the same manner as in Example 1.

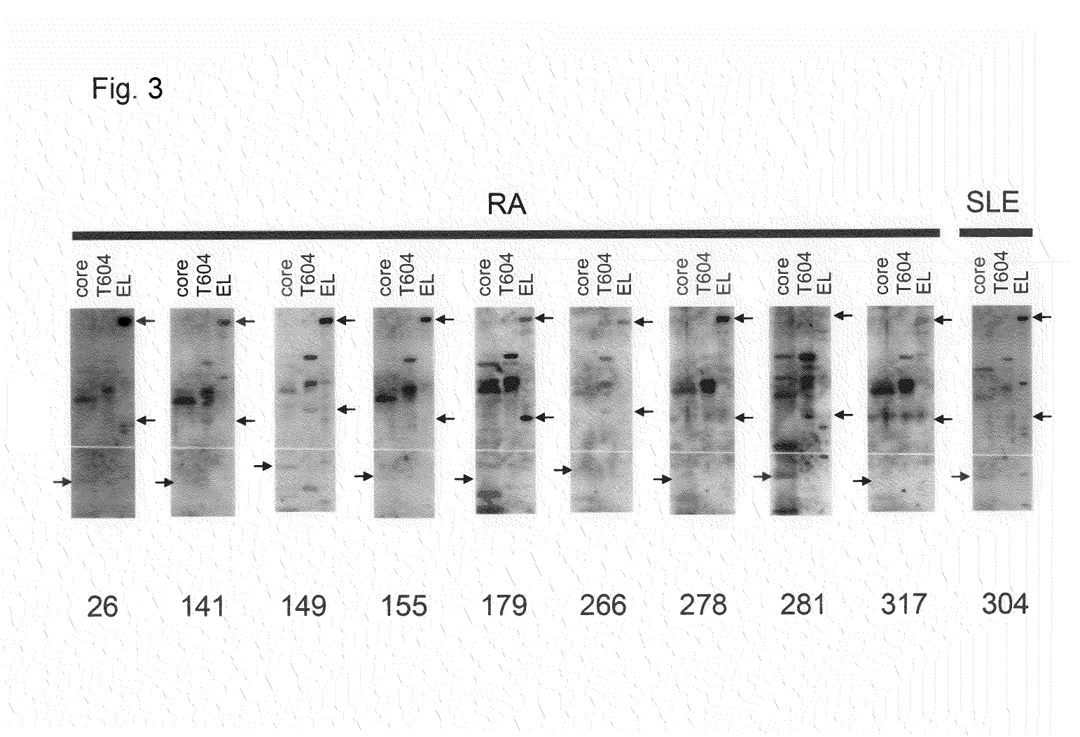

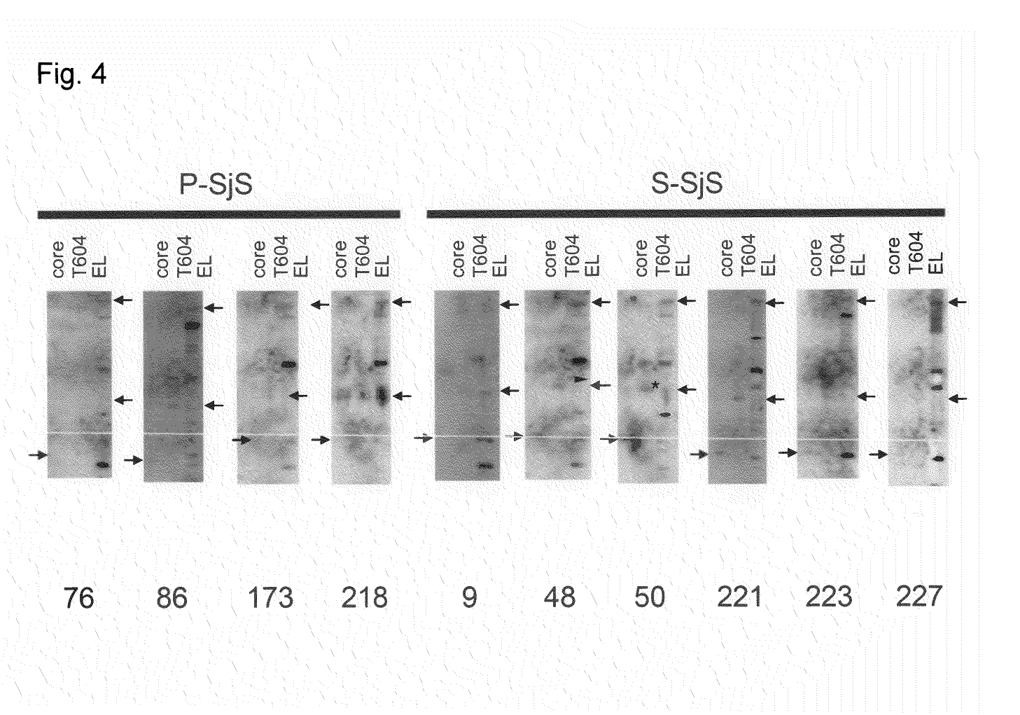

The results are shown in FIGS. 3 and 4. FIGS. 3 and 4 each show representative photographs of immunoblot analysis using serum samples obtained from patients. In FIGS. 3 and 4, positions of bands with EL, T604, and core are indicated by arrows, and bands at different positions are all non-specific bands. The serum sample numbers are shown below the immunoblot photographs.

FIG. 3 shows 10 serum samples that had strongly reacted with IP₃R2. These serum samples included 9 from patients with RA and 1 from a patient with SLE. All samples except for the serum sample No. 281 generated strong bands in lanes containing EL (amino acid residues 1 to 2171). In contrast, no significant band was detected in lanes containing core and T604 (FIG. 3).

FIG. 4 shows 10 serum samples that had strongly reacted with IP₃R1. These serum samples included 4 from patients with P-SjS and 6 from patients with S-SjS. Most serum samples had weakly reacted with all of EL, T604, and core, and their cognate epitope was considered to be present in the core protein (FIG. 4).

This suggests that the produced autoantibodies would recognize different types and/or epitopes of IP₃R proteins, depending on the type of autoimmune disease. In the present example, relatively extensive regions of the three domains were employed as antigens. Since epitopes recognized by antibodies each consist of about 5 or 6 amino acids, use of a narrower region as an antigen may enable thorough analysis of the correlation between a particular epitope region and an autoimmune disease type.

### INDUSTRIAL APPLICABILITY

The present invention provides a reagent for detecting an autoantibody. Such reagent enables easy detection of an anti-inositol 1,4,5-trisphosphate receptor (IP₃R) antibody in a sample and thus is effective for detecting an autoantibody in a sample. Also, a kit comprising such reagent detects an autoantibody in a sample to thereby diagnose an autoimmune disease. Thus, such kit is effective for early diagnosis and monitoring of autoimmune diseases.

All publications, patents, and patent applications cited herein are incorporated herein by reference in their entirety.

1. A reagent for detecting an autoantibody comprising a component selected from the group consisting of an inositol 1,4,5-trisphosphate receptor (IP₃R) protein, a fragment thereof and a mixture thereof.

2. The reagent according to claim 1, wherein IP₃R is a mouse or human IP₃R.

3. The reagent according to claim 1, wherein IP₃R is at least one selected from the group consisting of type 1 IP₃R (IP₃R1), type 2 IP₃R (IP₃R2), and type 3 IP₃R (IP₃R3).

4. The reagent according to claim 1, wherein the IP₃R protein fragment comprises an amino acid selected from the group consisting of amino acids 224 to 604 of IP₃R1 or IP₃R2, amino acids 1 to 604 of IP₃R1 or IP₃R2, amino acids 1 to 2217 of IP₃R1, and amino acids 1 to 2171 of IP₃R2.

5. The reagent according to claim 1, wherein the IP₃R protein and/or a fragment thereof is immobilized on a solid phase.

6. The reagent according to claim 1, wherein the IP₃R protein and/or a fragment thereof is labeled.

7. A method for detecting an autoantibody comprising detecting an anti-inositol 1,4,5-trisphosphate receptor (IP₃R) antibody in a sample.

8. The method according to claim 7 comprising bringing a sample into contact with the IP₃R protein and/or a fragment thereof and detecting an anti-IP₃R antibody in the sample by assaying the reaction between the antibody and the IP₃R protein or a fragment thereof.

9. A diagnostic kit for an autoimmune disease comprising the reagent for detecting an autoantibody according to claim 1.

10. The diagnostic kit according to claim 9, wherein the autoimmune disease is selected from the group consisting of rheumatoid arthritis (RA), systemic lupus erythematodes (SLE), Sjogren's syndrome (SjS), systemic sclerosis (SSc), mixed connective tissue disease (MCTD), unclassified connective tissue disease (UCTD), polymyositis (PM), dermatomyositis (DM), Hashimoto's disease, primary biliary cirrhosis (PBC), ulcerative colitis, Crohn's disease, and Behcet's disease.

11. The diagnostic kit according to claim 9, wherein the reagent for detecting an autoantibody comprises at least one IP₃R protein and/or a fragment thereof selected from the group consisting of full-length IP₃R1, full-length IP₃R2, full-length IP₃R3, and a fragment comprising amino acids 224 to 604 of IP₃R1 or IP₃R2, amino acids 1 to 604 of IP₃R1 or IP₃R2, and amino acids 1 to 2217 of IP₃R1 or amino acids 1 to 2171 of IP₃R2.

12. The diagnostic kit according to claim 9, which further comprises a reagent for detecting at least one autoantibody selected from the group consisting of an anti-SS-A/Ro antibody, an anti-S-B/La antibody, an anti-U1RNP antibody, an anti-Sm antibody, an anti-Scl70 antibody, an anti-Ki antibody, an anti-Ku antibody, an anti-rRNP antibody, an anti-Wa antibody, an anti-p95c/p97/VCP antibody, an anti-centromere antibody, an anti-nuclear antibody, and a rheumatoid factor.
